SEQUENCE LISTING (60 sequences)

SEQ ID NO: 1 - DNA, 3921 bp, Homo sapiens
catgccttatgcaagagacctcagtcccccggaacaactcgatttccttccaatagaggtctgaggtggactcccacctcccttcgtgaagagttccctcctctcccccttcctaagaaagtcgatcttggctctatttgtgtcttatgttcatcaccctcattcctccggagaaagccgggttggtttatgtctttatttattcccggggccaagacgtccggaacctgtggctgcgcagacccggcactgataggcgaagacggagagaaatttacctcccgccgctgccccccagccaaacgtgacagcgcgcgggccggttgcgtgactcgtgacgtctccaagtcctataggtgcagcggctggtgagatagtcgctatcgcctggttgcctctttattttactggggtatgcctggtaataaacagtaatatttaatttgtcggagaccacaaaccaaccttgagctgggaggtacgtgctcttcttgacagacgttggaagaagacctggcctaaagaggtctcttttggtggtccttttcaaagtcttcacctgagccctgctctccagcgaggcgcactcctggcttttgcgctccaaagaagaggtgggatagttggagagcagaaccttgcgcgggcacagggccctgggcgcaccatggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagctaaagtgaggcctgcttcacaacagacatttcctagagaaagagagagagagaggagaaagagagagaaggagagagacagtagccaagagaaccccacggacaagatttttcatttcacccaatgttcacatctgcactcaaggtcgctggatgctgatctaatcagtagcttgaaaccacaattttaaaaatgtgactttcttgttttgtctcaaaacttaaaaaaacaaacacaaaaagatgagtcccaccccccactaccaccacacccatcaaccagccacattcacgctactccccagatctcttcccccattccttcttttgggctctagaaagtcttgcctcattgagtgtttttccctagtgcgtagttggagtctgtccctgtcttggtgttaatgttgacattgttatataataaatgataatatatttttttctttcaattttcttaatgggacccagtcccttatttggggggaggtctgaggcaagtatatttcaaaatatgtacttgcgggattcccttcaagtaaaccatccctgaaacctaaattcacgtttccccttgactaagaaaagcacctacctctgccatgtgatgtttctgaaaagcctctgtatgtccccatttgctttggttttgtcctgccttctccaatatcacgtgctcagttttgcctctacttacccatggagtcaggataacactgacgctccctggcatcctatcttattcagccctaccatcttgccagctctgtctttccagctgtctgtcgctaaaacgtggcctatagcttcccttccggaaagcttgctttgaaaaacttaaaaagcccccgtttacatgtaggcaggactgtgataacagtgcaagctctgtgttgacaagagttgtggacaaaaagccaaaataaatattcttcctgattaaaaaaattttttttgaaaaaaacaaggccagccccaaccttccaaacctccatcaccaacaacccaaactggatgtcaagcaaaatgcacaattcctacagaagaggcaagacacagtcaccaatgatatctcgccaaagaaaccacgcccacaccaatgccaacacaaaactgtgtttactgaaagccgaaaacagtattaaaaaaagtgtgtaagtaaagtgttatggtagggttcttcagatgtaatattttactggtactatttatttataaataggaattctaattaagtaataacatgaaatgaaacccagcataggagctggccaagagcttttaattttattgatactcaaaaccaagtttgtgtttttttgtttttttttgtttttttcctctttcgaatgtgctttgctttttttgattaaaaagaattttttttttccttttttataaacagaccctaataaagagaacagggtaagatgtgaggctgagtgtgtttaagtacgtgagagagtgtgagtgtgtttgtaagtgagtgtccctatgcgattatgtctctttacgttgctaaggggggagggtgaggattaagtactcgtgccttatatttgtgtgccaattaatgcctaataaataccatgtgcttaaacaagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 518 aa, Homo sapiens
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 3 - DNA, 6449 bp, Homo sapiens
gagctgttctccttttcccattcgtcttctgtcacttccttcctggacgcagttttctggacgagtctggttacttttaatccgaccggccgctgagagccactttctcctcctcctcctcctcctccttctcttcctcctccttcttcctcctcctcctcctcttccgagcggcctcggcgcgcgcgaatgcgcggccccgcgccccccccctcgcgcgcgctcccctcgcgcgcgcgcacacacgcacacatcgtctccagctctctgctcgctctgctcgcagtcacagacacttgagcacacgcgtacacccagacatcttcgggctgctattggattgactttgaaggttctgtgtgggtcgccgtggctgcatgtttgaatcaggtggagaagcacttcaacgctggacgaagtaaagattattgttgttattttttttttctctctctctctctcttaagaaaggaaaatatcccaaggactaatctgatcgggtcttccttcatcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatattaaaaaaaaaaaatctatcaaccatttcatatatatcccactactcaaggtatccatggaacatgaaagaataacatttatgcagaggaaaaacaaaaacatccctgaaaatatacacactcatacacacacacgcacaggggaataaaataagaaaatcattttcctcaccatagacttgatcccatccttacaacccatccttctaacttgatgtgtataaaatatgcaaacatttcacaaatgttctttgtcatttcaaaatactttagtatatcaatatcagtagataccagtgggtgggaaagggtcattacatgaaaatatgaagaaatagccatattagttttttaacctgcaatttgcctcagcaacaaagaaaaagtgaatttttaatgctgaagataaagtaagctaaagtaccagcagaagccttggctatttatagcagttctgacaatagttttataagaacatgaagagaacagaatcacttgaaaatggatgccagtcatctcttgttcccactactgaattcttataaagtggtggcaagatagggaagggataatctgagaatttttaaaagatgatttaatgagaagaagcacaattttgattttgatgagtcactttctgtaaacaatcttggtctatctttacccttataccttatctgtaatttaccatttattgtatttgcaaagctagtatggtttttaatcacagtaaatcctttgtattccagactttagggcagagccctgagggagtattattttacataacccgtcctagagtaacattttaggcaacattcttcattgcaagtaaaagatccataagtggcattttacacggctgcgagtattgttatatctaatcctattttaaaagatttttggtaatatgaagcttgaatactggtaacagtgatgcaatatacgcaagctgcacaacctgtatattgtatgcattgctgcgtggaggctgtttatttcaacctttttaaaaattgtgttttttagtaaaatggcttattttttcccaaaggtggaatttagcattttgtaatgatgaatataaaaatacctgtcatccccagatcatttaaaagttaactaaagtgagaatgaaaaaacaaaattccaagacactttttaaaagaatgtctgccctcacacacttttatggatttgtttttcttacatacccatcttttaacttagagatagcattttttgccctctttattttgttgtttgtttctccagagagtaaacgctttgtagttctttctttaaaaaacattttttttaaagaagaagaagccacttgaaccctcaataaaggctgttgcctaagcatggcatacttcatctgttctcatttgtgccatctgccgtgatgtcgtcacttttatggcgttaatttcctgccactacagatcttttgaagattgctggaatactggtgtctgttagaatgcttcagactacagatgtaattaaaggcttttcttaatatgttttaaccaaagatgtggagcaatccaagccacatatcttctacatcaaatttttccattttggttattttcataatctggtattgcattttgccttccctgttcatacctcaaattgattcatacctcagtttaattcagagaggtcagttaagtgacggattctgttgtggtttgaatgcagtaccagtgttctcttcgagcaaagtagacctgggtcactgtaggcataggacttggattgcttcagatggtttgctgtatcatttttcttctttttcttttcctggggacttgtttccattaaatgagagtaattaaaatcgcttgtaaatgagggcatacaagcatttgcaacaaatattcaaatagaggctcacagcggcataagctggactttgtcgccactagatgacaagatgttataactaagttaaaccacatctgtgtatctcaagggacttaattcagctgtctgtagtgaataaaagtgggaaattttcaaaagtttctcctgctggaaataaggtataatttgtattttgcagacaattcagtaaagttactggctttcttagtgaaaaaaaaaaa

SEQ ID NO: 4 - protein, 473 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 5 - DNA, 3473 bp, Homo sapiens
agtcagcgccctagggccgagttgctgggccggggacccgagccgcgagctggggacttggaggcggccggcgcaggggccgcgagaggcttcgtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttcaaaaaa

SEQ ID NO: 6 - protein, 442 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 7 - DNA, 2368 bp, Homo sapiens
ctgtacatggagatcttgctgggaaaatccgcttgctcccctcacgtcgtccagcccaggagaaccaccgccgtcaccccggagcttcctcggccaccgcgcagagccctccgagagcccgagccgcggtcttcgagctccaaggctcattcagggccccagatccttgccccgaaaggagaggatctgagaaaatggatgcactgagacctctctgaaaaccctccgagagagcgcgagaggagcgaggacacgttactcgcagctaaaatcacatttaaggaccaaaacaacaacaaccaaaaatttcattaaaacaataagcgcccaagaacccagatcgggctggtggggggaggggaagaggcgggaaggggagggtcgcacggaggtagctttgcagtgagcagtcgaccccgccgccccccggcacagctggaccggctcctccagccgcggctcagactcgcccctggattccgggttagcttcggtgccaggaccgcggcccgggcttggattcccgagactccgcgtaccagcctcgcgggagccccggcacctttgtatgagcacgagaggattctgcctccgcgcagcagcccgggaagcaggagccgaagcgcgggccgtggagcaaggcgggaaccggaggcggcggcggcggcggccaggggcgcacggtgccaggaccagctcgccgcgccccatggggagccggcggccgcagcgctgctgaggcgggcccggctggccaggcggggggacggggcccgggctgcagcagccccctctgcggctgccgggcgggcccgggcgcccgggggctggggggtggggggtgggggaggacgccgagcgctgaggcaggggcccgggccgagggcgcggcggggctgcgcgcacgctggggcgcgtggaggggcgcggagggcgaaatgagtctggtaggtggttttccccaccacccggtggtgcaccacgagggctacccgtttgccgccgccgccgccgcagctgccgccgccgccgccagccgctgcagccatgaggagaacccctacttccatggctggctcatcggccaccccgagatgtcgccccccgactacagcatggccctgtcctacagccccgagtatgccagcggcgccgccggcctggaccactcccattacgggggggtgccgccgggcgccgggcccccgggcctgggggggccgcgcccggtgaagcgccgaggcaccgccaaccgcaaggagcggcgcaggactcagagcatcaacagcgccttcgccgaactgcgcgagtgcatccccaacgtacccgccgacaccaaactctccaaaatcaagaccctgcgcctggccaccagctacatcgcctacctcatggacctgctggccaaggacgaccagaatggcgaggcggaggccttcaaggcagagatcaagaagaccgacgtgaaagaggagaagaggaagaaggagctgaacgaaatcttgaaaagcacagtgagcagcaacgacaagaaaaccaaaggccggacgggctggccgcagcacgtctgggccctggagctcaagcagtgaggaggaggagaaggaggaggaggagagcgcgagtgagcaggggccaaggcgccagatgcagacccaggactccggaaaagccgtccgcgctccgctctgaggactccttgcatttggaatcatccggtttatttatgtgcaatttccttcccctctctttgaccccctttgaggcatctgctccccgtctccccctccaaaaaaaaagtggatatttgaagaaaagcattccatattttaatacgaagaggacactcccgtgtggtaagggatcccgtcgtctcatagattctgtgtgcgtgaatgttccctcttggctgtgtagacaccagcgttgccccccgccaacctactcaaccccttccagataaagacagtgggcactagtgcgtttgtgaagtgtatctttaatacttggcctttggatataaatattcctgggtattataaagttttatttcaaagcagaaaacagggccgctaacatttccgttggggtcggtatctagtgctatccattcatctgtggtcgttccctctttgaagatgtttccaacagccacttgttttgtgcacttccgtcctctaaaactaaatggaatttaattaatattgaaggtgtaaacgttgtaagtattcaataaaccactgtgttttttttttacaaaaaccttaatcttttaatggctgatacctcaaaagagttttgaaaacaaagctgttatacttgttttcgtaatatttaaaatattcagaagtaaactaaattatcatga

SEQ ID NO: 8 - protein, 217 aa, Homo sapiens
MSLVGGFPHHPVVHHEGYPFAAAAAAAAAAAASRCSHEENPYFHGWLIGHPEMSPPDYSMALSYSPEYASGAAGLDHSHYGGVPPGAGPPGLGGPRPVKRRGTANRKERRRTQSINSAFAELRECIPNVPADTKLSKIKTLRLATSYIAYLMDLLAKDDQNGEAEAFKAEIKKTDVKEEKRKKELNEILKSTVSSNDKKTKGRTGWPQHVWALELKQ

SEQ ID NO: 9 - protein, 23 aa, Artificial sequence
  Synthetic polypeptide
GALFLGWLGAAGSTMGAKKKRKV

SEQ ID NO: 10 - protein, 16 aa, Artificial sequence
  Synthetic polypeptide
RQIKIWFQNRRMKWKK

SEQ ID NO: 11 - protein, 7 aa, Artificial sequence
  Synthetic polypeptide
RRMKWKK

SEQ ID NO: 12 - protein, 16 aa, Artificial sequence
  Synthetic polypeptide
RRWRRWWRRWWRRWRR

SEQ ID NO: 13 - protein, 18 aa, Artificial sequence
  Synthetic polypeptide
RGGRLSYSRRRFSTSTGR

SEQ ID NO: 14 - protein, 11 aa, Artificial sequence
  Synthetic polypeptide
YGRKKRRQRRR

SEQ ID NO: 15 - protein, 9 aa, Artificial sequence
  Synthetic polypeptide
RKKRRQRRR

SEQ ID NO: 16 - protein, 11 aa, Artificial sequence
  Synthetic polypeptide
YARAAARQARA

SEQ ID NO: 17 - protein, 8 aa, Artificial sequence
  Synthetic polypeptide
RRRRRRRR

SEQ ID NO: 18 - protein, 8 aa, Artificial sequence
  Synthetic polypeptide
KKKKKKKK

SEQ ID NO: 19 - protein, 27 aa, Artificial sequence
  Synthetic polypeptide
  misc_feature (25)..(25): Xaa can be any naturally occurring amino acid
GWTLNSAGYLLGKINLKALAALAKXIL

SEQ ID NO: 20 - protein, 18 aa, Artificial sequence
  Synthetic polypeptide
LLILLRRRIRKQANAHSK

SEQ ID NO: 21 - protein, 16 aa, Artificial sequence
  Synthetic polypeptide
SRRHHCRSKAKRSRHH

SEQ ID NO: 22 - protein, 11 aa, Artificial sequence
  Synthetic polypeptide
NRARRNRRRVR

SEQ ID NO: 23 - protein, 15 aa, Artificial sequence
  Synthetic polypeptide
RQLRIAGRRLRGRSR

SEQ ID NO: 24 - protein, 13 aa, Artificial sequence
  Synthetic polypeptide
KLIKGRTPIKFGK

SEQ ID NO: 25 - protein, 10 aa, Artificial sequence
  Synthetic polypeptide
RRIPNRRPRR

SEQ ID NO: 26 - protein, 18 aa, Artificial sequence
  Synthetic polypeptide
KLALKLALKALKAALKLA

SEQ ID NO: 27 - protein, 14 aa, Artificial sequence
  Synthetic polypeptide
KLAKLAKKLAKLAK

SEQ ID NO: 28 - protein, 27 aa, Artificial sequence
  Synthetic polypeptide
GALFLGFLGAAGSTNGAWSQPKKKRKV

SEQ ID NO: 29 - protein, 21 aa, Artificial sequence
  Synthetic polypeptide
KETWWETWWTEWSQPKKKRKV

SEQ ID NO: 30 - protein, 20 aa, Artificial sequence
  Synthetic polypeptide
LKKLLKKLLKKLLKKLLKKL

SEQ ID NO: 31 - protein, 34 aa, Artificial sequence
  Synthetic polypeptide
QAATATRGRSAASRPTERPRAPARSASRPRRPVE

SEQ ID NO: 32 - protein, 23 aa, Artificial sequence
  Synthetic polypeptide
MGLGLHLLVLAAALQGAKSKRKV

SEQ ID NO: 33 - protein, 26 aa, Artificial sequence
  Synthetic polypeptide
AAVALLPAVLLALLAPAAANYKKPKL

SEQ ID NO: 34 - protein, 28 aa, Artificial sequence
  Synthetic polypeptide
MANLGYWLLALFVTMWTDVGLCKKRPKP

SEQ ID NO: 35 - protein, 24 aa, Artificial sequence
  Synthetic polypeptide
LGTYTQDFNKFHTFPQTAIGVGAP

SEQ ID NO: 36 - protein, 26 aa, Artificial sequence
  Synthetic polypeptide
  misc_feature (24)..(24): Xaa can be any naturally occurring amino acid
DPKGDPKGVTVTVTVTVTGKGDPXPD

SEQ ID NO: 37 - protein, 14 aa, Artificial sequence
  Synthetic polypeptide
PPPPPPPPPPPPPP

SEQ ID NO: 38 - protein, 18 aa, Artificial sequence
  Synthetic polypeptide
VRLPPPVRLPPPVRLPPP

SEQ ID NO: 39 - protein, 10 aa, Artificial sequence
  Synthetic polypeptide
PRPLPPPRPG

SEQ ID NO: 40 - protein, 30 aa, Artificial sequence
  Synthetic polypeptide
SVRRRPRPPYLPRPRPPPFFPPRLPPRIPP

SEQ ID NO: 41 - protein, 21 aa, Artificial sequence
  Synthetic polypeptide
TRSSRAGLQFPVGRVHRLLRK

SEQ ID NO: 42 - protein, 23 aa, Artificial sequence
  Synthetic polypeptide
GIGKFLHSAKKFGKAFVGEIMNS

SEQ ID NO: 43 - protein, 37 aa, Artificial sequence
  Synthetic polypeptide
KWKLFKKIEKVGQNIRDGIIKAGPAVAVVGQATQIAK

SEQ ID NO: 44 - protein, 28 aa, Artificial sequence
  Synthetic polypeptide
ALWMTLLKKVLKAAAKAALNAVLVGANA

SEQ ID NO: 45 - protein, 26 aa, Artificial sequence
  Synthetic polypeptide
GIGAVLKVLTTGLPALISWIKRKRQQ

SEQ ID NO: 46 - protein, 14 aa, Artificial sequence
  Synthetic polypeptide
INLKALAALAKKIL

SEQ ID NO: 47 - protein, 33 aa, Artificial sequence
  Synthetic polypeptide
GFFALIPKIISSPLPKTLLSAVGSALGGSGGQE

SEQ ID NO: 48 - protein, 15 aa, Artificial sequence
  Synthetic polypeptide
LAKWALKQGFAKLKS

SEQ ID NO: 49 - protein, 27 aa, Artificial sequence
  Synthetic polypeptide
  misc_feature (23)..(23): Xaa can be any naturally occurring amino acid
SMAQDIISTIGDLVKWIIQTVNXFTKK

SEQ ID NO: 50 - protein, 41 aa, Artificial sequence
  Synthetic polypeptide
LLGDFFRKSKEKIGKEFKRIVQRIKQRIKDFLANLVPRTES

SEQ ID NO: 51 - protein, 18 aa, Artificial sequence
  Synthetic polypeptide
PAWRKAFRWAWRMLKKAA

SEQ ID NO: 52 - protein, 18 aa, Artificial sequence
  Synthetic polypeptide
KLKLKLKLKLKLKLKLKL

SEQ ID NO: 53 - DNA, 2794 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 54 - protein, 480 aa, Homo sapiens [sequence omitted]

SEQ ID NO: 55 - DNA, 4904 bp, Homo sapiens
agacactgcgccgcggctgcgcagtacacgggaggcttttaattccattgtggagaggacggcgttatttttattaactggaggcgacggcggctgcggcggcggcgggacccgcctcctccggggtatgaaaatcggcagtgggttcctgagtggcggcggaggtaccggcagtagcggtggtagcggctccggcggcggtggtagtggcggcggcggcggcggcggcagcagcggcaggagggcagagatggaacccacctttccccagggtatggttatgttcaaccaccgtcttcccccggtcaccagcttcacccggccggcggggtcggccgcccctcccccgcaatgcgtgttatcctcctctacctccgcagccccggccgctgagcccccccctccgccagccccggacatgactttcaagaaggagccggcggcgtcagccgcggccttcccctcgcagaggacctcctgggggttcttgcagtctttggttagcatcaaacaggagaaacccgcggatcctgaggagcagcagtcccaccaccaccatcaccaccaccactatggggggctgttcgctggagctgaagagaggtctccaggcctaggaggcggtgaaggggggagtcacggcgtcatccaggacctcagtattctccaccagcatgtccagcagcaaccagcccagcaccaccgtgacgtattactcagcagcagtagcaggactgatgaccaccatggcactgaggagccaaagcaggacactaatgtcaaaaaggcaaaaaggccaaagccagaatctcagggaatcaaagccaagaggaagccaagtgcatcttccaaaccttctttggttggagatggagaaggtgccatcctctccccaagtcagaaacctcatatctgtgatcactgtagtgctgctttccgaagctcctatcacctgcggagacatgtcctcattcatacaggagaaagacctttccagtgcagccagtgtagtatgggtttcattcagaaatacctactacagagacatgagaaaattcatagtagagagaagccatttggatgtgatcagtgcagcatgaagtttattcagaagtaccatatggagagacacaagaggacacatagtggagaaaagccatataagtgtgacacttgccaacagtatttttcaaggactgatagattgttgaagcacaggcgcacatgtggtgaagtcatagttaaaggagccactagtgcagaacctgggtcatcaaaccataccaatatgggtaatctggctgtgttgtctcagggaaatacaagttcttcaaggagaaaaacaaagtcaaaaagcatagctattgaaaataaggaacagaagaccggtaaaacaaatgaatcgcaaatttcaaataatataaacatgcagagttactcagtagaaatgcctaccgtgtcttccagtggaggcataattggcactggaatagatgaactgcagaagagggtgccaaaattgatctttaagaaaggaagcagaaagaatacagataaaaactaccttaactttgtgtcaccattaccagacatagtaggacagaaatccttgtctggaaaaccaagtggctcacttggcatagtatcaaataatagtgtggagaccattggtcttctccaaagtacaagtggcaaacaaggtcagataagtagtaattatgatgatgccatgcagttttcaaagaaaagaagatatttaccaactgccagcagcaacagtgccttttctataaacgtaggacacatggtctcccaacagtctgtcattcagtctgcaggtgtcagtgttttggacaatgaggcaccattgtcacttattgactcctcagctctaaatgctgaaattaaatcttgtcatgacaagtctggaattcctgatgaggttttacaaagtattttggatcaatactccaacaaatcagaaagccagaaagaggatcctttcaatattgcagaaccacgagtggatttacacacctcaggagaacactcagaattggttcaagaagaaaatttgagcccaggcacccaaacaccttcaaatgataaagcaagtatgttgcaagaatactccaaatacctccaacaggcttttgaaaaatccactaatgcaagttttactcttggacacggtttccaatttgtcagtttgtcttcacctctccacaaccacactttgtttccagaaaaacaaatatacactacgtctcctttggagtgtggtttcggccaatctgttacctcagtgttgccatcttcattgccaaagcctccttttgggatgttgtttggatctcagccaggtctttatttgtctgctttggatgctacacatcagcagttgacaccttcccaggagctggatgatctgatagattctcagaagaacttagagacttcatcagccttccagtcctcatctcagaaattgactagccagaaggaacagaaaaacttagagtcttcaacaggctttcagattccatctcaggagttagctagccagatagatcctcagaaagacatagagcctagaacaacgtatcagattgagaactttgcacaagcgtttggttctcagtttaagtcgggcagcagggtgccaatgacctttatcactaactctaatggagaagtggaccatagagtaaggacttcagtgtcagatttctcagggtatacaaatatgatgtctgatgtaagtgagccatgtagtacaagagtaaagacacccaccagccagagttacaggtaaggtcccaaaagtggccaggctggaggtcttctaatgtaattttgttttattttgagaacactgccattggaatgtttctacacgatcctattaagaataatgtgatgccctttcaatgcaacttttcatatttagtttattttgttagcgtgattttagctctgtttgtattatgatttttaatcaaaatcaatagattaaaaatagtttgacattcaaagtgacaatgtttagcaatcaaatttacatgtatagatcgtcagggaatagcccaaatgttttaaacgcaaaaaaaaagacaaaaaaaaaccaaaaaaaaaaaacctacaaaaaaaactttgttgctaggattaaggttattctaattgctttactctcaggaaagtgtaataacgcatgggaattctgtacgttatcactgtaatggaatatccaatttacagatagtatgatatacatttcatcatttaagtaagggatcgaaaacatttcaaattgctctatctgggctgatagacatttcgtcatttaagtaagggatcgaagacatttcaaattgctatctccatctgggctgatccaaaattctgagattgttggctacctatattttgttgcagcttttaaatgtactctgaacttccaaaccacattcattccagcctggtagaacaaatattcttggatctttgatcaaagcctggaatgatagctttaataaaagaaggaaaaaaaaaaagcaccctctcttgatctcaactgtaacaaggctgtaattccatcaagcgattctactgacatctagtacagtgttgaagcccaagtggttggctggttccagtattagaactcaagttgaaattatatgattaactgaaatttggggcatgcattatttattatacatggcctacaacggttctgtctgcgctgttacgtattacctacacttctgggtttttttgttcttgttctcagcctctctttaagagttcttgggccttccttttccctctttctattttaacagttttaattttagaaccatttacattccagtaggtaattcactatgcgctttatatgccttagggcatccaaataaaaaacaatagggtaaacatccccaaaacccacctttaaaaaaaaaaaaacccatacagatttccatatgtaataaaaccccgtgttcttggatagatattaataaggttgtaattccaactggtgacataactcctgtgagggataagaatgtgtctgcttatagtaataacatcagaacctctttgtgaaattgcggcctttaatgtacattttaatgaaagggtgcattagtattgtacaggggtcttgtaaagtcatcaaaacttgctatgaactctaattgccatttgaagctactgattggcagtggctctactctaaaactgctttttagcaattgtatttttttcctgattatattttttaatgtactttgatgtttatgctgatgagttttttatgtacttttggtgatgtttcagtcttatgtactcttctgacgtcagaaaagtaccactggttgtttgcagtcttattgtgtaatcagcctaccgcaggcttccatgtataataaagtaattaatattgtgcaagtgtaaaacacttctgttataaaagcagtgtttggaaaataagaaatattaaaaatggttacaaagtattagttaatttccttttcccactttcctccctttagctttagcagctaaagggtcaccaacttgtcattgcaagattcttagaaaatgtacttaatcttaaatacgctgagtactaaaaagtgtttatttcttatttcagtaagtgcggtaagttctattgagtacagagtacaagctgtaatcaaaggatggagaggacctttaccactgtatctcttaagctgtatgatttttctccagtttgttttatacaggttttgaatttctttaacttttattgtgtgtactgaatactgcatatgtattattctgattgtttgctctagtttactaccaataaaagtcctgttaatcacaagattaaaaaaaaaaaaaaaaaa

SEQ ID NO: 56 - protein, 895 aa, Homo sapiens
MKIGSGFLSGGGGTGSSGGSGSGGGGSGGGGGGGSSGRRAEMEPTFPQGMVMFNHRLPPVTSFTRPAGSAAPPPQCVLSSSTSAAPAAEPPPPPAPDMTFKKEPAASAAAFPSQRTSWGFLQSLVSIKQEKPADPEEQQSHHHHHHHHYGGLFAGAEERSPGLGGGEGGSHGVIQDLSILHQHVQQQPAQHHRDVLLSSSSRTDDHHGTEEPKQDTNVKKAKRPKPESQGIKAKRKPSASSKPSLVGDGEGAILSPSQKPHICDHCSAAFRSSYHLRRHVLIHTGERPFQCSQCSMGFIQKYLLQRHEKIHSREKPFGCDQCSMKFIQKYHMERHKRTHSGEKPYKCDTCQQYFSRTDRLLKHRRTCGEVIVKGATSAEPGSSNHTNMGNLAVLSQGNTSSSRRKTKSKSIAIENKEQKTGKTNESQISNNINMQSYSVEMPTVSSSGGIIGTGIDELQKRVPKLIFKKGSRKNTDKNYLNFVSPLPDIVGQKSLSGKPSGSLGIVSNNSVETIGLLQSTSGKQGQISSNYDDAMQFSKKRRYLPTASSNSAFSINVGHMVSQQSVIQSAGVSVLDNEAPLSLIDSSALNAEIKSCHDKSGIPDEVLQSILDQYSNKSESQKEDPFNIAEPRVDLHTSGEHSELVQEENLSPGTQTPSNDKASMLQEYSKYLQQAFEKSTNASFTLGHGFQFVSLSSPLHNHTLFPEKQIYTTSPLECGFGQSVTSVLPSSLPKPPFGMLFGSQPGLYLSALDATHQQLTPSQELDDLIDSQKNLETSSAFQSSSQKLTSQKEQKNLESSTGFQIPSQELASQIDPQKDIEPRTTYQIENFAQAFGSQFKSGSRVPMTFITNSNGEVDHRVRTSVSDFSGYTNMMSDVSEPCSTRVKTPTSQSYR

SEQ ID NO: 57 - DNA, 6950 bp, Homo sapiens
aatcgccggcagcctatgacatcagacaggaacgcctgggatgccgcgctgctcctggccaacctccgaggaggaggagggtcccgccggctaagagttaattagccccgcacggcgaggggggaggcgccagttttctggggacactggctgccactgtactcctacccaggggagctcacggagagttggatgaattctgggttgttagctgcggtcagctgggctcccgggagcctgttgctggtggagaacagggggcgcctggccaagggaccagcggcttgctgagactcaacatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtagaatgcccaatgcaccagtgctatggaagaccaatggagttccatgggggaaagcacacagccatacatactttactgtccaaaaacagaagaagaagaagagaattaaaaagaagcaatgatttctgtgccaatgaacaagaacaaaagtcatttttagaaatacatatactgtaatatttaccaacagtcagtaactgttaatgatttcaacaatgcattaaaagaatgtgctttctcagattaaggatgccaaaaaagatatttcactgccttttcaaagaccagtatattttctagcccataatttttctcaggcattgttggggcataagctcacactgtaagcttttctcatgaattcactagacataacgtggaaggaaaacgtagtcttttgggagtacagggaagccagcccctcaaagcttatggaagacatacctgcaatggaagctgttgcccaatgtctccattactatctttcaaaagagaagccagacccagcttcagatcaaaagttcttgagacagaggaacaaaaccaatcgatttccagggaagctaatcaactctcttttccctctaccacaaaactgccctgctggagtggttctgaacctgtacccaggactcgatgtggtcactaataacaattaacctgaactgagtccacagaactccactcggaactttcttcttttttaactagtggcccaatcattcccaccatctctgtgctgataagtacgtgtcctagatgagaaccctgaagaatgcagaccttcttcccccgaaggagatgccacaagctctccaacacagccccctttagttccaaagactagagatgaccacattggtagaagtatatctcgaggcacaggaagggagccccaccagggataattcagacaggactagagaataacatcatttcacataccctgggataaacaccctgggttcctatagaaggactattacttatgggagtccaacttctccttttgttttgttattatcagtttatctttctcccactccacttttccttcaaggtaccaatcctttcctgttcctcgtttggccatctttctttttctgcctccacattgggaggggaggacttctcagttctaacaagctgccatactcctaagaaagccatttttgaaaaatttaacaatccaggttcttctggagaactcattctccacacgcacagtttgctgcaaaaggaagttgcaagaatttcttgaggaagaaactggtgacttggtccatcagtcacgaagttctttctattctcgtttagttttcaagaaattattggtttgtgttgctctggggaaattggaaatcattacattgtaaagacaaatatggatgatatttacaagagagaatttcagatctgggtttttgaaagaaaacagaattgcgcattgaaaacgatggaaggaaaaagacaatggtctaatgtgcattcctcattacctctcgtggctttggctgggagttggaaaaagctaaaatttcagaacagtctctgtaaggctctctgtggctccagttcaccattttatattgttgcatgctgtagaaaggagctattgctgttgttttgtttttttatttaaatcactaaggcactgtttttatcttttgtaaaaaaaaaaaaaaagttgttcactgtgcacttatagaaaaaataatcaaaaatgttgggattttagaagctctctttttgataaaccaaagatttagaagtcattccattgttaacttgtaaaaatgtgtgaacacagagagtttttggtgattgctactctgaaagctgccagatcttattctgggggtgggatgtggaggaatacacatacacacacaaacatacatgtatgtataatagatatatacatatgtgtatattatatctgtgtgtgcatgtatctccaaaagcggcgttacagagttctacaccaaaagcctttaacccttaatctgctgtgaatgatacctggcctttctcactatgaatttctgattaaccaaccagactacacgttgcctctctgtgtatgactaacggctccaacccgatgactcacagctacttgcttatcgtgaacaagctcatcttggcaatgaatatggatgtgaaaagacagaacagcttcaccattagtagctggaaatggtatcacagtctcttatagaggaatatgaaaggaacaagaaaatcattttacattccttttatctgtattgtgctttaaaagatccacatggtaaattttttattttgcttttatgtcagtcatcagaaccaaaaaaatccagaagaaaaaattgccagtgtttcctttgaagatgaagctactggggaagaaaaccttattaatacactccacacatttgttcattcctcagctgttggtgttttcttggggtcttgacaaagcttgctggtcagtgcacttttcaggtgtcacgttttgctgtttgtatgttttttcttccccttacttcctttggaaaacaaactcacacagtgcccctactctgagacctgggactgagtgttaattattttttccttgggtatttctatctgagagactagacctagttaggaggcctctgtacttctccagattgtacctttttatggggatctttgaggctatgacccaggactgatagatatgccttacggaagacaaaagataaaatggttcctatatcctaatgcaaaccaacacagttaaaagagcagatctctggataactgctctcaacctgcttctacagtctccacaaaccgcattcaccctctctcttcatagctcagacatgaaatttgagggagaaaactggagataattgggagaaaattgatgaagttggctgcttccagtagatcagataatccatgaatttgtctcccattgagaattttattttaaattcttttaaactcttcgttgtgtcttttgtgatgacaaatcaggcatgactaaaagatgtacagagacttacgaagatggtcacattcaagttccctaatgctcttagaacctgaagatgaccatgtgtagttttcttaagacctctgaacccccatggtgatgaagacttgaagacatttgcagctatctgctgcagtctggtagattcatacttatctaaagaagtcaaaaaatttattcgtgcaagtgcttgcaggaagccagtgcttattagtagtgaccctgcttctatcaacgttattgagacaacacatattctattctaagggagaaagagggaggaagagagggagggagggaggaagaagagggagggagcgaggaaggaagataggagatgggtaggggggtaaagagaaaaggagggagaagggaaggaaggaaagaagagaggaaagaaaggagggaaggaaaaaagggccaaactttctgatctatgaacttctcagttcagctgtcacattatgagaagtaaatcagaatttttttaaggagaagtcattcttagcactacaataattgtaccagtaattgaggaaaccaagacaatcttcacctgaataatagagggtctgagaactgtcagccttttgccattcaaaaacatttatgtccaacctgaaaaaaaagcatcaataaaacctatcccaagcattc

SEQ ID NO: 58 - protein, 986 aa, Homo sapiens
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 59 - DNA, 2490 bp, Artificial sequence
  Synthetic oligonucleotide
agcactctctcacttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctcctcgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgcctttttttttcttactgttttattacaaacttacaaaaatatgtataaccctgttttatacaaactagtttcgtaataaaactttttcctttttttaaaatgaaaataaaaaaaa

SEQ ID NO: 60 - protein, 236 aa, Artificial sequence
  Synthetic polypeptide
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

(19) United States
(12) Patent Application Publication
(10) Pub. No.: US 2021/0180023 A1
(43) Pub. Date: Jun. 17, 2021

(54) CARDIAC REPAIR BY REPROGRAMMING OF ADULT CARDIAC FIBROBLASTS INTO CARDIOMYOCYTES

(71) Applicant: The Board of Regents of the University of Texas System, Austin, TX (US)

(72) Inventors: Eric N. OLSON, University Park, TX (US); Huanyu ZHOU, Redwood City, TX (US); Rhonda BASSEL-DUBY, Dallas, TX (US)

(73) Assignee: The Board of Regents of the University of Texas System, Austin, TX (US)

(21) Appl. No.: 16/639,523

(22) PCT Filed: Jun. 4, 2018
(86) PCT No.: PCT/US18/35830

§ 371 Date: Feb. 14, 2020

Related U.S. Application Data
(60) Provisional application No. 62/545,700, filed on Aug. 15, 2017.

(51) Int. Cl. C12N 5/077; C12N 9/12; C07K 14/47; A61K 45/06; A61P 9/10

(52) U.S. Cl. CPC C12N 5/0657; C12N 9/12; C12Y 207/11001; A61K 45/06; A61P 9/10; C07K 14/4702

(57) ABSTRACT

The present disclosure involves the use of reprogramming factors including AKT1, GATA4, TBX5, MEF2C, HAND2 and either ZNF281 or AS-CL1 to reprogram adult non-cardiomyocytes, such as cardiac fibroblasts into cardiomyocytes, both in vitro and in vivo. Such methods find particular use in the treatment of patients post-myocardial infarction to prevent or limit scarring and to promote myocardial repair.

### PRIORITY CLAIM

The present application claims benefit of priority to U.S. Provisional Application Ser. No. 62/545,700, filed Aug. 15, 2017, the entire contents of which are hereby incorporated by reference.

The invention was made with government support under grant no. 1U01 HL100401-01 awarded by the National Institutes of Health (NHLBI). The government owns certain rights in the invention.

### INCORPORATION OF SEQUENCE LISTING

The sequence listing that is contained in the file named “UTFDP3268WO_ST25.txt”, which is 93 KB (as measured in Microsoft Windows®) and was created on Jun. 4, 2018, is filed herewith by electronic submission and is incorporated by reference herein.

### BACKGROUND

### 1. Field

The present disclosure relates generally to the fields of cardiology, developmental biology and molecular biology. More particularly, it concerns gene regulation and cellular physiology in cardiomyocytes. Specifically, the invention relates to the use of various transcription factors to reprogram cardiac fibroblasts into cardiomyocytes and the use of such factors in the prevention of scarring and repair in post-myocardial infarction.

### 2. DESCRIPTION OF RELATED ART

A heart attack, also known as myocardial infarction (MI), occurs when the flow of blood to the heart is obstructed. Following a massive MI, the human heart can lose hundreds of millions of cardiomyocytes. Due to the limited capacity of the heart to regenerate, the lost cardiomyocytes are replaced by scar tissue, thus impairing contractility of a large portion of the heart muscle. Clinical interventions following a heart attack have improved dramatically over the past decades (Jessup and Brozena, 2003). However, due to the inability of the heart to replenish lost cardiomyocytes, MI remains the primary cause of death in the world (Jessup and Brozena, 2003; Xin et al., 2013). Approximately one third of the cells within the adult mouse heart are fibroblasts, which upon injury are activated and contribute to the formation of scar tissue (Tallquist and Molkentin, 2017).

Reprogramming cardiac fibroblasts (CFs) to induced-cardiomyocytes (iCMs) by forced expression of cardiac transcription factors represents a potential means of enhancing cardiac repair by reducing scar tissue while simultaneously generating new cardiomyocytes (Fu et al., 2013; Ieda et al., 2010; Nam et al., 2013; Qian et al., 2012; Song et al., 2012). However, low efficiency as well as the lack of understanding of the molecular basis of the reprogramming process represent challenges to its potential clinical application (Kojima and Ieda, 2017; Srivastava and DeWitt, 2016; Vaseghi et al., 2017).

The first cardiac reprogramming cocktail consisted of three cardiac transcription factors, GATA4, MEF2C, and TBX5 (GMT) (Ieda et al., 2010). Subsequent effort has been directed toward optimization of cardiac reprogramming by generating different cocktails that contain various combinations of proteins, microRNAs, and small molecules (Abad et al., 2017; Ifkovits et al., 2014; Mohamed et al., 2017; Muraoka et al., 2014; Song et al., 2012; Wang et al., 2015; Yamakawa et al., 2015; Zhao et al., 2015; Zhou et al., 2015; Zhou et al., 2016). Most of these studies adopted a candidate approach to examine the effects of supplementing the GMT cocktail with other key cardiac factors or regulators that have been shown to promote other somatic cell reprogramming processes. For example, adding the cardiac transcription factor HAND2, which plays an essential role in cardiac morphogenesis, enhanced the cardiac reprogramming efficiency of GMT both in vitro and in vivo (Song et al., 2012). Addition of a cardiac microRNA, miR-133, was also shown to enhance cardiac reprogramming in mouse and human fibroblasts (Muraoka et al., 2014; Nam et al., 2013). In addition, SB431542, a TGF-β inhibitor, and DAPT, a Notch inhibitor, which have been shown to promote Induced pluripotent stem cell (iPSC) reprogramming, can enhance cardiac reprogramming (Abad et al., 2017; Ifkovits et al., 2014). However, these reprogramming cocktails are still relatively inefficient and the molecular basis of the reprogramming process is poorly understood.

The inventors previously took an unbiased approach to screen a library of protein kinases and discovered that AKT1 dramatically accelerates and amplifies the cardiac reprogramming process (Zhou et al., 2015). The optimal cocktail, which contains AKT1, GATA4, HAND2, MEF2C, and TBX5 (which the inventors refer to as AGHMT), converts ˜50% of mouse embryonic fibroblasts (MEFs) to iCMs (Zhou et al., 2015). However, the efficiency of conversion of adult tail-tip fibroblasts (TTFs) is less than 1%. Since heart attacks primarily occur in adults, inefficient reprogramming of adult fibroblasts to iCMs diminishes the clinical translatability of this technique (Zhou et al., 2015). Thus, understanding how to apply this reprogramming approach to adult fibroblasts would have a significant impact on cardiac therapies.

### SUMMARY

Thus, in accordance with the present disclosure, there is provided a method of reprogramming a cardiac fibroblast comprising contacting said cardiac fibroblast with AKT1, GATA4, TBX5, MEF2C, HAND2 and ZNF281. Contacting may comprise delivering AKT1, GATA4, TBX5, MEF2C, HAND2 and ZNF281 proteins to the cardiac fibroblast. One or more of AKT1, GATA4, TBX5, MEF2C, HAND2 and ZNF281 may comprise a heterologous cell permeability peptide (CPP). The method may further comprises contacting the cardiac fibroblast with an anti-inflammatory agent, and/or may further comprise contacting the cardiac fibroblast with myocardin.

Contacting may instead comprise delivering AKT1, GATA4, TBX5, MEF2C, HAND2 and ZNF281expression cassettes to the cardiac fibroblasts MEF2C, HAND2. The expression cassettes may be comprised in one or more replicable vectors, such as viral vectors, such as adeno-associated virus (AAV), non-integrated lentivirus, adenoviral vectors or retroviral vectors. The one or more replicable vectors may also be non-viral vectors, such as those disposed in a lipid delivery vehicle. The method may further comprise contacting a cardiac fibroblast with an anti-inflammatory agent, and/or may further comprise contacting the cardiac fibroblast with a myocardin expression cassette.

In another embodiment, there is provided a method of treating a subject having suffered a myocardial infarct (MI) comprising delivering to the subject AKT1, GATA4, TBX5, MEF2C, HAND2 and ZNF281. Contacting may comprise delivering AKT1, GATA4, TBX5, MEF2C, HAND2 and ZNF281 proteins to the cardiac fibroblast. One or more of AKT1, GATA4, TBX5, MEF2C, HAND2 and ZNF281 may comprise a heterologous cell permeability peptide (CPP). The method may further comprises contacting the cardiac fibroblast with an anti-inflammatory agent, and/or may further comprise contacting the cardiac fibroblast with myocardin.

Contacting may instead comprise delivering AKT1, GATA4, TBX5, MEF2C, HAND2 and ZNF281expression cassettes to the cardiac fibroblasts MEF2C, HAND2. The expression cassettes may be comprised in one or more replicable vectors, such as viral vectors, such as adeno-associated virus (AAV), non-integrated lentivirus, adenoviral vectors or retroviral vectors. The one or more replicable vectors may also be non-viral vectors, such as those disposed in a lipid delivery vehicle. The method may further comprise contacting a cardiac fibroblast with an anti-inflammatory agent, and/or may further comprise contacting the cardiac fibroblast with a myocardin expression cassette.

AKT1, GATA4, TBX5, MEF2C, HAND2 and ZNF281 proteins or expression cassettes may be delivered 24 hours to one month following the MI. At least one of the AKT1, GATA4, TBX5, MEF2C, HAND2 and ZNF281 proteins may be delivered multiple times, or at least one of the AKT1, GATA4, TBX5, MEF2C, HAND2 and ZNF281 expression cassettes may be delivered multiple times. At least one of AKT1, GATA4, TBX5, MEF2C, HAND2 and ZNF281 proteins may be delivered 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or at least one of AKT1, GATA4, TBX5, MEF2C, HAND2 and ZNF281 expression cassettes may be delivered 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times. AKT1, GATA4, TBX5, MEF2C, HAND2 and ZNF281 or expression cassette coding therefor may be delivered daily. The method may further comprise delivering myocardin or myocardin expression cassette daily.

AKT1, GATA4, TBX5, MEF2C, HAND2 and ZNF281 proteins or expression cassettes may be delivered via intracardiac injection. The subject may be further administered oxygen, aspirin, and/or nitroglycerin. The subject may be further administered percutaneous coronary intervention. The subject may be further administered a fibrinolytic. The MI is non-ST-elevated MI, or ST-elevated MI.

In yet another embodiment, there is provided a method preventing or delaying development of cardiac hypertrophy or heart failure in a subject having suffered a myocardial infarct (MI) comprising providing to the subject AKT1, GATA4, TBX5, MEF2C, HAND2 and ZNF281 proteins or expression cassettes coding therefor. The method may further comprising administering to the subject a secondary anti-hypertrophic or heart failure therapy, such as a PKD inhibitor, a beta blocker, an ionotrope, a diuretic, ACE-I, All antagonist, BNP, a Ca′-blocker, or an HDAC inhibitor. Preventing or delaying may comprise preventing or delaying cardiac hypertrophy, such as preventing or delaying comprises preventing or delaying one or more of decreased exercise capacity, decreased cardiac ejection volume, increased left ventricular end diastolic pressure, increased pulmonary capillary wedge pressure, decreased cardiac output or cardiac index, increased pulmonary artery pressures, increased left ventricular end systolic and diastolic dimensions, increased left and right ventricular wall stress, increased wall tension, decreased quality of life, and/or increased disease related morbidity or mortality.

AKT1, GATA4, TBX5, MEF2C, HAND2 and ZNF281 proteins may be administered to the subject, or AKT1, GATA4, TBX5, MEF2C, HAND2 and ZNF281 expression cassettes may be administered to the subject. The method may further comprise administering myocardin protein or expression cassette coding therefore to the subject, and/or further comprise administering an anti-inflammatory agent to the subject.

In still another embodiment, there is provided a method of reducing a decrease in exercise tolerance of a subject having suffered a myocardial infarction comprising administering to the subject AKT1, GATA4, TBX5, MEF2C, HAND2 and ZNF281 proteins or expression cassettes coding therefor. The method may further comprise administering myocardin or an expression cassette coding therefor to the subject.

In still a further embodiment, there is provided a method of reducing hospitalization of a subject having suffered a myocardial infarction comprising administering to the subject AKT1, GATA4, TBX5, MEF2C, HAND2 and ZNF281 proteins or expression cassettes coding therefor. The method may further comprise administering myocardin or an expression cassette coding therefor to the subject.

In another embodiment, there is provided a method of improving quality of life of a subject having suffered a myocardial infarction comprising administering to the subject AKT1, GATA4, TBX5, MEF2C, HAND2 and ZNF281 proteins or expression cassettes coding therefor. The method may further comprise administering myocardin or an expression cassette coding therefor to the subject.

In an additional embodiment, there is provided a method of decreasing morbidity of a subject having suffered a myocardial infarction comprising administering to the subject AKT1, GATA4, TBX5, MEF2C, HAND2 and ZNF281 proteins or expression cassettes coding therefor. The method may further comprise administering myocardin or an expression cassette coding therefor to the subject.

In yet an additional embodiment, there is provided a method of decreasing mortality of a subject having suffered a myocardial infarction comprising administering to the subject AKT1, GATA4, TBX5, MEF2C, HAND2 and ZNF281 proteins or expression cassettes coding therefor. The method may further comprising administering myocardin or an expression cassette coding therefor to the subject.

In a further embodiment, there is provided a method of reprogramming a cardiac fibroblast comprising contacting said cardiac fibroblast with AKT1, GATA4, TBX5, MEF2C, HAND2 and ASCL1. Contacting may comprise delivering AKT1, GATA4, TBX5, MEF2C, HAND2 and ASCL1 proteins to the cardiac fibroblast. One or more of AKT1, GATA4, TBX5, MEF2C, HAND2 and ASCL1 may comprise a heterologous cell permeability peptide (CPP). The method may further comprises contacting the cardiac fibroblast with an anti-inflammatory agent, and/or may further comprise contacting the cardiac fibroblast with myocardin.

Contacting may instead comprise delivering AKT1, GATA4, TBX5, MEF2C, HAND2 and ASCL1 expression cassettes to the cardiac fibroblasts MEF2C, HAND2. The expression cassettes may be comprised in one or more replicable vectors, such as viral vectors, such as adeno-associated virus (AAV), non-integrated lentivirus, adenoviral vectors or retroviral vectors. The one or more replicable vectors may also be non-viral vectors, such as those disposed in a lipid delivery vehicle. The method may further comprise contacting a cardiac fibroblast with an anti-inflammatory agent, and/or may further comprise contacting the cardiac fibroblast with a myocardin expression cassette.

In another embodiment, there is provided a method of treating a subject having suffered a myocardial infarct (MI) comprising delivering to the subject AKT1, GATA4, TBX5, MEF2C, HAND2 and ASCL1. Contacting may comprise delivering AKT1, GATA4, TBX5, MEF2C, HAND2 and ASCL1 proteins to the cardiac fibroblast. One or more of AKT1, GATA4, TBX5, MEF2C, HAND2 and ASCL1 may comprise a heterologous cell permeability peptide (CPP). The method may further comprises contacting the cardiac fibroblast with an anti-inflammatory agent, and/or may further comprise contacting the cardiac fibroblast with myocardin.

Contacting may instead comprise delivering AKT1, GATA4, TBX5, MEF2C, HAND2 and ASCL1 expression cassettes to the cardiac fibroblasts MEF2C, HAND2. The expression cassettes may be comprised in one or more replicable vectors, such as viral vectors, such as adeno-associated virus (AAV), non-integrated lentivirus, adenoviral vectors or retroviral vectors. The one or more replicable vectors may also be non-viral vectors, such as those disposed in a lipid delivery vehicle. The method may further comprise contacting a cardiac fibroblast with an anti-inflammatory agent, and/or may further comprise contacting the cardiac fibroblast with a myocardin expression cassette.

AKT1, GATA4, TBX5, MEF2C, HAND2 and ASCL1 proteins or expression cassettes may be delivered 24 hours to one month following the MI. At least one of the AKT1, GATA4, TBX5, MEF2C, HAND2 and ASCL1 proteins may be delivered multiple times, or at least one of the AKT1, GATA4, TBX5, MEF2C, HAND2 and ASCL1 expression cassettes may be delivered multiple times. At least one of AKT1, GATA4, TBX5, MEF2C, HAND2 and ASCL1 proteins may be delivered 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or at least one of AKT1, GATA4, TBX5, MEF2C, HAND2 and ASCL1 expression cassettes may be delivered 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times. AKT1, GATA4, TBX5, MEF2C, HAND2 and ASCL1 or expression cassette coding therefor may be delivered daily. The method may further comprise delivering myocardin or myocardin expression cassette daily.

AKT1, GATA4, TBX5, MEF2C, HAND2 and ASCL1 proteins or expression cassettes may be delivered via intracardiac injection. The subject may be further administered oxygen, aspirin, and/or nitroglycerin. The subject may be further administered percutaneous coronary intervention. The subject may be further administered a fibrinolytic. The MI is non-ST-elevated MI, or ST-elevated MI.

In yet another embodiment, there is provided a method preventing or delaying development of cardiac hypertrophy or heart failure in a subject having suffered a myocardial infarct (MI) comprising providing to the subject AKT1, GATA4, TBX5, MEF2C, HAND2 and ASCL1 proteins or expression cassettes coding therefor. The method may further comprising administering to the subject a secondary anti-hypertrophic or heart failure therapy, such as a PKD inhibitor, a beta blocker, an ionotrope, a diuretic, ACE-I, All antagonist, BNP, a Ca′-blocker, or an HDAC inhibitor. Preventing or delaying may comprise preventing or delaying cardiac hypertrophy, such as preventing or delaying comprises preventing or delaying one or more of decreased exercise capacity, decreased cardiac ejection volume, increased left ventricular end diastolic pressure, increased pulmonary capillary wedge pressure, decreased cardiac output or cardiac index, increased pulmonary artery pressures, increased left ventricular end systolic and diastolic dimensions, increased left and right ventricular wall stress, increased wall tension, decreased quality of life, and/or increased disease related morbidity or mortality.

AKT1, GATA4, TBX5, MEF2C, HAND2 and ASCL1 proteins may be administered to the subject, or AKT1, GATA4, TBX5, MEF2C, HAND2 and ASCL1 expression cassettes may be administered to the subject. The method may further comprise administering myocardin protein or expression cassette coding therefore to the subject, and/or further comprise administering an anti-inflammatory agent to the subject.

In still another embodiment, there is provided a method of reducing a decrease in exercise tolerance of a subject having suffered a myocardial infarction comprising administering to the subject AKT1, GATA4, TBX5, MEF2C, HAND2 and ASCL1 proteins or expression cassettes coding therefor. The method may further comprise administering myocardin or an expression cassette coding therefor to the subject.

In still a further embodiment, there is provided a method of reducing hospitalization of a subject having suffered a myocardial infarction comprising administering to the subject AKT1, GATA4, TBX5, MEF2C, HAND2 and ASCL1 proteins or expression cassettes coding therefor. The method may further comprise administering myocardin or an expression cassette coding therefor to the subject.

In another embodiment, there is provided a method of improving quality of life of a subject having suffered a myocardial infarction comprising administering to the subject AKT1, GATA4, TBX5, MEF2C, HAND2 and ASCL1 proteins or expression cassettes coding therefor. The method may further comprise administering myocardin or an expression cassette coding therefor to the subject.

In an additional embodiment, there is provided a method of decreasing morbidity of a subject having suffered a myocardial infarction comprising administering to the subject AKT1, GATA4, TBX5, MEF2C, HAND2 and ASCL1 proteins or expression cassettes coding therefor. The method may further comprise administering myocardin or an expression cassette coding therefor to the subject.

In yet an additional embodiment, there is provided a method of decreasing mortality of a subject having suffered a myocardial infarction comprising administering to the subject AKT1, GATA4, TBX5, MEF2C, HAND2 and ASCL1 proteins or expression cassettes coding therefor. The method may further comprising administering myocardin or an expression cassette coding therefor to the subject.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. Identification of activators and inhibitors of AGHMT-mediated cardiac reprogramming from a human ORF cDNA screen. (FIG. 1A) Schematic diagram of the human ORF cDNA library screen strategy for cardiac reprogramming in adult tail-tip fibroblasts (TTFs). (FIG. 1B) Venn diagram showing the number of activators identified from the screen. Genes with Z-scores of αMHC-GFP or cTnT expression ≥2 were defined as activators. 25 genes induced αMHC expression only, 35 genes induced cTnT expression only and 11 genes induced expression of both markers. (FIG. 1C) Venn diagram showing the number of inhibitors identified from the screen. Genes with Z-scores of αMHC or cTnT expression ≤−2 were defined as inhibitors. 121 genes repressed αMHC expression only, 41 genes repressed cTnT expression only and 33 genes repressed both αMHC and cTnT expression. (FIG. 1D) Representative immunocytochemistry images of TTFs from adult αMHC-GFP transgenic mice treated with 5F and either empty virus or viruses encoding activators (ZNF281 or PHF7) and inhibitors (FOXA3 or SOX9). Cells were fixed and stained for αMHC-GFP (green), cTnT (red), and DAPI (blue) 9 days post-infection. 5F: AGHMT. (Scale bars: 2 mm). (FIG. 1E) Pathways enriched in activators (n=49) and inhibitors (n=129), respectively, by DAVID Pathway Analysis.

FIGS. 2A-D. Anti-inflammatory drugs promote cardiac reprogramming. (FIG. 2A) 5F-reprogrammed TTFs treated with either DMSO or the indicated anti-inflammatory drugs for 7 days post-infection show reduced expression of inflammatory genes (IL6, Ccl2, and Ptgs1), but increased expression of cardiac genes (Myh6, Actc1 and Nppa). Dex, Dexamethasone (10 μM); Nab, Nabumetone (10 μM). (FIG. 2B) Immunocytochemistry images of 5F-reprogrammed adult αMHC-GFP transgenic TTFs treated with DMSO or the indicated anti-inflammatory drugs for 7 days show that anti-inflammatory drugs enhance expression of cardiac markers with 5F. αMHC-GFP (green), cTnT (red), DAPI (blue). Scale bars: 500 μm. (FIGS. 2C-D) Representative flow cytometry plot (FIG. 2C) and analyses (FIG. 2D) of αMHC-GFP+ and cTnT+ cells in 5F-reprogrammed adult αMHC-GFP transgenic TTFs treated with DMSO or the indicated anti-inflammatory drugs for 7 days show that anti-inflammatory drugs increase the percentage of reprogrammed cells in addition to 5F. *P<0.05

FIGS. 3A-D. ZNF281 enhances cardiac reprogramming of adult fibroblasts. (FIG. 3A) Immunocytochemistry images of adult αMHC-GFP transgenic TTFs 7 days postinfection with Empty, ZNF281, 5F, or 6F (5F+ZNF281) retroviruses show that ZNF281 enhances expression of cardiac markers with 5F. αMHC-GFP (green), cTnT (red), DAPI (blue). Scale bars: 500 μm. (FIGS. 3B-C) Representative flow cytometry plot (FIG. 3B) and analyses (FIG. 3C) of αMHC-GFP+ and cTnT+TTFs 7 days post-infection with Empty, 5F, or 6F retrovirus shows that ZNF281 increases the percentage of reprogrammed cells with 5F. (FIG. 3D) Transcript levels of cardiac marker genes (Myh6 and Actc1) and fibroblast marker genes (Cola2 and Sox9) were increased or decreased, respectively, a week after induction by adding ZNF281 to 5F in TTFs. * P<0.05.

FIGS. 4A-E. RNA-seq analysis shows that ZNF281 enhances cardiac genes and represses inflammatory genes. (FIG. 4A) Heat map of 1,500 differentially expressed genes in 5F versus 6F treated TTFs identified by RNA-seq. Red indicates up-regulation, and blue indicates down-regulation. RNA-seq samples were prepared from adult TTFs reprogrammed for 7 days. (FIGS. 4B-C) Gene ontology analysis showing biological processes associated with unregulated genes (FIG. 4B) and down-regulated genes (FIG. 4C) by ZNF281. (FIGS. 4D-E) Gene expression changes between 6F and 5F for selected cardiac markers (FIG. 4D) or inflammatory markers (FIG. 4E) as determined by RNA-seq.

FIGS. 5A-B. ZNF281 represses the inflammatory response through the NuRD complex. (FIG. 5A) TTFs were infected with Empty, 5F plus Empty, ZNF281 or each individual NuRD complex subunit retroviruses for 7 days. Transcript levels of inflammatory (Il6, and Ccl2) and cardiac (Myh6 and Actc1) marker genes were determined by q-PCR. (FIG. 5B) Representative flow cytometry plot of αMHC-GFP+ and cTnT+TTFs 7 days post-infection with Empty, 5F plus Empty, ZNF281 or each individual NuRD complex subunit retroviruses.

FIGS. 6A-F. ZNF281 interacts with GATA4 to synergistically activate cardiac genes. (FIG. 6A) Co-immunoprecipitation assays were performed using HEK293 cells transfected with equal amounts of plasmid DNA encoding Myc-tagged GATA4, HAND2, MEF2c, or TBX5 and/or Flag-tagged ZNF281. IP, immunoprecipitation; IB, immunoblot. (FIG. 6B) Luciferase reporter assays were performed using HEK293 cells transfected with equal amounts of ZNF281 and/or GATA4 expression plasmids, as indicated, along with αMHC-luciferase reporter plasmid. (FIG. 6C) Heat maps showing ChIP-seq data for H3K27ac in adult mouse heart, and ZNF281 and GATA4 binding in reprogrammed TTFs at ±5 kB around the peak center. ChIP-seq experiments were performed using TTFs infected with 6F for 2 days. (FIG. 6D) ZNF281 binding motif enriched within ZNF281 binding peaks. (FIG. 6E) GATA4 binding motif enriched within GATA4 binding peaks. (FIG. 6F) Venn diagram showing the number of overlapping peaks between Heart H3K27ac, ZNF281 and GATA4. *P<0.05

FIGS. 7A-F. GATA4 recruits ZNF281 to cardiac enhancers. (FIGS. 7A-B) Heat map for ZNF281 or GATA4 genomic binding at ±2 kb around the peak center in each cluster. ChIP-seq experiments were performed using adult TTFs reprogrammed for 2 days with 6F, 6F-G(GATA4) or 6F-Z(ZNF281). (FIG. 7C) Integrative Genomics Viewer (IGV) browser tracks at chr1:137,694,960-137,749,970 (mm9) show an example of peaks that belong to each indicated clusters. (FIGS. 7D-E) Gene ontologies (FIG. 7D) and pathways (FIG. 7E) enriched in genes that associate with each indicated cluster identified by gene ontology enrichment analysis and pathway analysis. (FIG. 7F) Model showing the mechanism of action of ZNF281 on AGHMT-mediated direct cardiac reprogramming. ZNF281 is a cardiac transcription coactivator, recruited by GATA4 to cardiac enhancers to activate cardiac gene expression. ZNF281 also represses the inflammatory response, which acts as a barrier pathway to cardiac reprogramming.

FIG. 8. ZNF281 mRNA expression profile. Transcript levels of ZNF281 in different tissues isolated from adult wild-type C57BL6 mice, as determined with quantitative PCR.

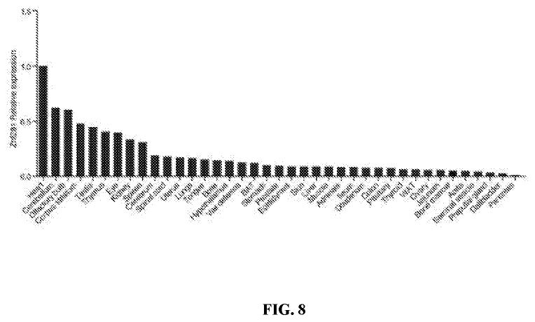

FIGS. 9A-B. ZNF281 represses the inflammatory response through the NuRD complex. (FIG. 9A) Representative flow cytometry plot of TTFs 7 days post-infection with Empty, 5F plus Empty, ZNF281 or each individual NuRD complex subunit retroviruses. (FIG. 9B) Quantification of αMHC-GFP+ and cTnT+ cells from flow cytometry plots.

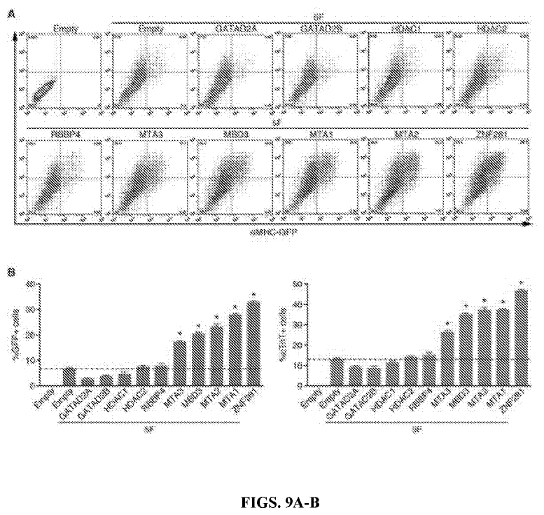

FIGS. 10A-D. ZNF281 enhances cardiac reprogramming of adult cardiac fibroblasts. (FIG. 10A) Immunocytochemistry images of adult αMHC-GFP transgenic CFs 7 days post-infection with Empty, ZNF281, 5F, or 6F (5F+ZNF281) retroviruses. αMHC-GFP (green), cTnT (red), DAPI (blue). Scale bars: 500 μm. (B-C) Representative flow cytometry plot (FIG. 10B) and analyses (FIG. 10C) of αMHC-GFP+ and cTnT+CFs 7 days post-infection with Empty, 5F, or 6F retrovirus. * P<0.05. (FIG. 10D) Percentage of beating cells, relative to the number of input cells after 4 weeks post-infection with Empty, ZNF281, 5F, or 6F (5F+ZNF281) retroviruses.

FIG. 11. Representative immunocytochemistry images of tail tip fibroblasts (TTFs) from adult αMHC-GFP transgenic mice treated with AGHMT and either empty virus or viruses encoding ASCL1. Cells were fixed and stained for αMHC-GFP (green), cTnT (red), and DAPI (blue) 9 days post-infection.

### DETAILED DESCRIPTION OF ILLUSTRATIVE EMBODIMENTS

Heart failure is one of the leading causes of morbidity and mortality in the world. In the U.S. alone, estimates indicate that 3 million people are currently living with cardiomyopathy and another 400,000 are diagnosed on a yearly basis. Heart disease and its manifestations, including coronary artery disease, myocardial infarction, congestive heart failure and cardiac hypertrophy, clearly presents a major health risk in the United States today. The cost to diagnose, treat and support patients suffering from these diseases is well into the billions of dollars.

One particularly severe manifestation of heart disease is myocardial infarction (MI). Typically, MI results from an acute thrombocytic coronary occlusion that occurs in a coronary artery as a result of atherosclerosis and causes myocardial cell death, i.e., an infarct. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic. Cardiac hypertrophy is an adaptive response of the heart to virtually all forms of cardiac disease, including myocardial infarction. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

Treatment with pharmacological agents still represents the primary mechanism for reducing or eliminating the manifestations of heart failure, including those resulting from MIs. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure. If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Given that most known regulators of cardiac development or iPSC reprogramming have been extensively tested in previous studies, and none were able to efficiently reprogram adult fibroblasts to iCMs, the inventors sought to identify previously unidentified regulators of cardiac reprogramming. Here, they describe their findings from an unbiased screen of 786 genes encoding transcription factors, epigenetic regulators, cytokines, and nuclear receptors to augment AGHMT-dependent cardiac reprogramming of adult TTFs. This screen led to the discovery of 49 activators and 129 inhibitors of cardiac reprogramming. These factors participate in various signaling pathways and biological processes, including the TGF-β and Notch signaling pathways, which have been shown to be important for cardiac reprogramming (Abad et al., 2017; Ifkovits et al., 2014). Interestingly, several factors involved in the inflammatory response were identified as inhibitors of cardiac reprogramming, indicating the inflammatory response might act as a barrier for adult cardiac reprogramming. Consistent with this notion, treatment of adult fibroblasts with anti-inflammatory drugs dramatically increased cardiac reprogramming efficiency. Many of these newly identified factors, including the strongest activator of cardiac reprogramming, Krüppel-Type Zinc-Finger Transcription Factor 281 (ZNF281), do not have a characterized function in cardiac development, and thus would not have been anticipated to impinge on the mechanisms of fibroblast-tocardiomyocyte reprogramming. The inventors show that ZNF281 enhances cardiac reprogramming by associating with GATA4 on cardiac enhancers and by inhibiting inflammatory signaling which antagonizes cardiac reprogramming. These results not only identify ZNF281 as a robust and efficient activator of adult cardiac reprogramming, but also provide new insights into the molecular mechanisms underlying cardiac reprogramming, cardiac development and cardiogenesis. These and other aspects of the disclosure are described in detail below.

### I. REPROGRAMMING FACTORS

A transcription factor (sometimes called a sequence-specific DNA-binding factor) is a protein that binds to specific DNA sequences, thereby controlling the movement (or transcription) of genetic information from DNA to mRNA. Transcription factors perform this function alone or with other proteins in a complex, by promoting (as an activator), or blocking (as a repressor) the recruitment of RNA polymerase (the enzyme that performs the transcription of genetic information from DNA to RNA) to specific genes.

A defining feature of transcription factors is that they contain one or more DNA-binding domains (DBDs), which attach to specific sequences of DNA adjacent to the genes that they regulate. Additional proteins such as coactivators, chromatin remodelers, histone acetylases, deacetylases, kinases, and methylases, while also playing crucial roles in gene regulation, lack DNA-binding domains, and, therefore, are not classified as transcription factors.

The present disclosure involves the inventors' observation that certain transcription factors can combine to reprogram adult cardiac fibroblasts into cardiomyocytes, and can do so in situ without the need for complicated ex vivo culturing steps and readministration. In particular, it is shown that AKT1+TBX5+MEF2C+HAND2+GATA4+ZNF281 contemplated, as well as the addition of other factors.

A. AKT1

RAC-alpha serine/threonine-protein kinase is an enzyme that in humans is encoded by the AKT1 gene. This enzyme belongs to the AKT subfamily of serine/threonine kinases that contain SH2 (Src homology 2-like) domains. It is commonly referred to as PKB, or by both names as “Akt/PKB.” AKT1 is catalytically inactive in serum-starved primary and immortalized fibroblasts. AKT1 and the related AKT2 are activated by platelet-derived growth factor. The activation is rapid and specific, and it is abrogated by mutations in the pleckstrin homology domain of AKT1. It was shown that the activation occurs through phosphatidylinositol 3-kinase. In the developing nervous system, AKT is a critical mediator of growth factor-induced neuronal survival. Survival factors can suppress apoptosis in a transcription-independent manner by activating the serine/threonine kinase AKT1, which then phosphorylates and inactivates components of the apoptotic machinery. Mice lacking Akt1 display a 25% reduction in body mass, indicating that AKT1 is critical for transmitting growth-promoting signals, most likely via the IGF1 receptor. A single-nucleotide polymorphism in this gene causes Proteus syndrome.

AKT1 (RAC-alpha serine/threonine-protein kinase); mRNA=NM_001014431 (SEQ ID NO: 53); Protein=NP_001014431 (SEQ ID NO: 54).

B. TBX5

T-box transcription factor TBX5 is a protein that in humans is encoded by the TBX5 gene. This gene is a member of a phylogenetically conserved family of genes that share a common DNA-binding domain, the T-box. T-box genes encode transcription factors involved in the regulation of developmental processes. This gene is closely linked to related family member T-box 3 (ulnar mammary syndrome) on human chromosome 12. The encoded protein may play a role in heart development and specification of limb identity. Mutations in this gene have been associated with Holt-Oram syndrome, a developmental disorder affecting the heart and upper limbs. Several transcript variants encoding different isoforms have been described for this gene. See Basson et al. (1997) and Terrett et al. (1994).

TBX5 (T-box 5); mRNA=NM_000192 (SEQ ID NO: 1); Protein=NP_000183 (SEQ ID NO: 2).

C. MEF2C

Myocyte-specific enhancer factor 2C also known as MADS box transcription enhancer factor 2, polypeptide C is a protein that in humans is encoded by the MEF2C gene. MEF2C is a transcription factor in the MEF2 family. The gene is located at 5q14.3 on the minus strand and is 200,723 bases in length. The encoded protein has 473 amino acids with a predicted molecular weight of 51.221 kD. Three isoforms have been identified. Several post translational modifications have been identified including phosphorylation on serine-59 and serine-396, sumoylation on lysine-391, acetylation on lysine-4 and proteolytic cleavage. The mature protein is found in the nucleus and the gene's expression is maximal in the post natal period.

MEF2C has been shown to interact with MAPK7, EP300, Sp1 transcription factor, TEAD1, SOX18, HDAC4, HDAC7 and HDAC9. This gene is involved in cardiac morphogenesis and myogenesis and vascular development. It may also be involved in neurogenesis and in the development of cortical architecture. Mice without a functional copy of the Mef2c gene die before birth and have abnormalities in the heart and vascular system. In human mutations of this gene have resulted in severe psychomotor retardation, periodic tremor and an abnormal motor pattern with mirror movement of the upper limbs observed during infancy, hypotonia, abnormal EEG, epilepsy, absence of speech, autistic behavior, bruxism, and mild dysmorphic features, mild thinning of the corpus callosum and delay of white matter myelination in the occipital lobes. See McDermott et al. (1993) and Molkentin et al. (1996).

MEF2C (myocyte enhancer factor 2C); mRNA=NM_002397 (SEQ ID NO: 3); Protein=NP_002388 (SEQ ID NO: 4).

D. GATA4

Transcription factor GATA-4 is a protein that in humans is encoded by the GATA4 gene. This gene encodes a member of the GATA family of zinc finger transcription factors. Members of this family recognize the GATA motif which is present in the promoters of many genes. This protein is thought to regulate genes involved in embryogenesis and in myocardial differentiation and function. Mutations in this gene have been associated with cardiac septal defects as well as reproductive defects. GATA4 has been shown to interact with NKX2-5, TBX5, ZFPM2, Serum response factor, HAND2 and HDAC2. See White et al. (1995).

GATA4 (GATA binding protein 4); mRNA=NM_002052 (SEQ ID NO: 5); Protein=NP_002043 (SEQ ID NO: 6).

E. HAND2

Heart- and neural crest derivatives-expressed protein 2 is a protein that in humans is encoded by the HAND2 gene. The protein encoded by this gene belongs to the basic helix-loop-helix family of transcription factors. This gene product is one of two closely related family members, the HAND proteins, which are asymmetrically expressed in the developing ventricular chambers and play an essential role in cardiac morphogenesis. Working in a complementary fashion, they function in the formation of the right ventricle and aortic arch arteries, implicating them as mediators of congenital heart disease. In addition, this transcription factor plays an important role in limb and branchial arch development. See Russell et al. (1999).

HAND2 (heart and neural crest derivatives expressed 2); mRNA=NM_021973 (SEQ ID NO: 7); Protein=NP_068808 (SEQ ID NO: 8).

F. ZNF281

Zinc finger protein 281 (ZNF281) is a transcription repressor that plays a role in regulation of embryonic stem cells (ESCs) differentiation. It is required for ESC differentiation and acts by mediating autorepression of NANOG in ESCs. It binds to the NANOG promoter and promotes association of NANOG protein to its own promoter and recruits the NuRD complex, which deacetylates histones. It does not appear to be required for establishment and maintenance of ESCs. ZNF281 represses the transcription of a number of genes including GAST, ODC1 and VIM, and binds to the G-rich box in the enhancer region of these genes.

ZNF281 (Zinc Finger Protein 281); mRNA=NM_012482 (SEQ ID NO: 55); Protein=NP_036614 (SEQ ID NO: 56).

G. ASCL1

Achaete-scute homolog 1 is a protein that in humans is encoded by the ASCL1 gene. Because it was discovered subsequent to studies on its homolog in Drosophila, the Achaete-scute complex, it was originally named MASH-1 for mammalian achaete scute homolog-1. ASCL1 has been shown to interact with Myocyte-specific enhancer factor 2A.

This gene encodes a member of the basic helix-loop-helix (BHLH) family of transcription factors. The protein activates transcription by binding to the E box (5′-CANNTG-3′). Dimerization with other BHLH proteins is required for efficient DNA binding. This protein plays a role in the neuronal commitment and differentiation and in the generation of olfactory and autonomic neurons. It is highly expressed in medullary thyroid cancer and small cell lung cancer and may be a useful marker for these cancers. The presence of a CAG repeat in the gene suggests that it may also play a role in tumor formation.

Development of the vertebrate nervous system begins when the neural tube forms in the early embryo. The neural tube eventually gives rise to the entire nervous system, but first neuroblasts must differentiate from the neuroepithelium of the tube. The neuroblasts are the cells that undergo mitotic division and produce neurons. Asc is central to the differentiation of the neuroblasts and the lateral inhibition mechanism which inherently creates a safety net in the event of damage or death in these incredibly important cells.

Differentiation of the neuroblast begins when the cells of the neural tube express Asc and thus upregulate the expression of Delta, a protein essential to the lateral inhibition pathway of neuronal commitment. Delta can diffuse to neighboring cells and bind to the Notch receptor, a large transmembrane protein which upon activation undergoes proteolytic cleavage to release the intracellular domain (Notch-ICD). The Notch-ICD is then free to travel to the nucleus and form a complex with Suppressor of Hairless (SuH) and Mastermind. This complex acts as transcription regulator of Asc and accomplishes two important tasks. First, it prevents the expression of factors required for differentiation of the cell into a neuroblast. Secondly, it inhibits the neighboring cell's production of Delta. Therefore, the future neuroblast will be the cell that has the greatest Asc activation in the vicinity and consequently the greatest Delta production that will inhibit the differentiation of neighboring cells. The select group of neuroblasts that then differentiate in the neural tube are thus replaceable because the neuroblast's ability to suppress differentiation of neighboring cells depends on its own ability to produce Asc. This process of neuroblast differentiation via Asc is common to all animals. Although this mechanism was initially studied in Drosophila, homologs to all proteins in the pathway have been found in vertebrates that have the same bHLH structure.

In addition to its important role in neuroblast formation, Asc also functions to mediate autonomic nervous system (ANS) formation. Asc was initially suspected to play a role in the ANS when ASCL1 was found expressed in cells surrounding the dorsal aorta, the adrenal glands and in the developing sympathetic chain during a specific stage of development. Subsequent studies of mice genetically altered to be MASH-1 deficient revealed defective development of both sympathetic and parasympathetic ganglia, the two constituents of the ANS.

ASCL1 (Achaete-scute homolog 1); mRNA=NM_004316 (SEQ ID NO: 59); Protein=NP_004307 (SEQ ID NO: 60).

H. Myocardin

Myocardin is a protein that in humans is encoded by the MYOCD gene. Myocardin is a smooth muscle and cardiac muscle-specific transcriptional coactivator of serum response factor. When expressed ectopically in nonmuscle cells, myocardin can induce smooth muscle differentiation by its association with serum response factor (SRF).

MYOCD (myocardin); mRNA=NM_001146312.1 (SEQ ID NO: 57); Protein=NP_001139784.1 (SEQ ID NO: 58).

### III. PROTEIN DELIVERY

The present disclosure, in one aspect, relates to the production and formulation of transcription factors as well as their delivery to cells, tissues or subjects. In general, recombinant production of proteins is well known and is therefore no described in detail here. The discussion of nucleic acids and expression vectors, found below, is however incorporated in this discussion.

A. Purification of Proteins

It will be desirable to purify proteins according to the present disclosure. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present disclosure concern the purification, and in particular embodiments, the substantial purification, of an encoded protein or peptide. The term “purified protein” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein composition that has been subjected to fractionation to remove various other components, and which composition substantially retains its expressed biological activity. Where the term “substantially purified” is used, this designation will refer to a composition in which the protein forms the major component of the composition, such as constituting about 50%, about 60%, about 70%, about 80%, about 90%, about 95% or more of the proteins in the composition.

Various methods for quantifying the degree of purification of the protein will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present disclosure is discussed below.

B. Cell Permeability Peptides

The present disclosure contemplates the use of a cell permeability peptide (also called a cell delivery peptide, or cell transduction domain) linked to transcription factors. Such domains have been described in the art and are generally characterized as short amphipathic or cationic peptides and peptide derivatives, often containing multiple lysine and arginine resides (Fischer, 2007). Other examples are shown in Table 1, below.

TABLE 1
CDD/CTD PEPTIDES

| | SEQ ID | | SEQ ID |
|---|---|---|---|
| | NO: | | NO: |
| GALFLGWLGAAGSTMGAKKKRK | 9 | QAATATRGRSAASRPTERPRAPARS | 31 |
| V | | ASRPRRPVE | |
| RQIKIWFQNRRMKWKK | 10 | MGLGLHLLVLAAALQGAKSKRKV | 32 |
| RRMKWKK | 11 | AAVALLPAVLLALLAPAAANYKKP | 33 |
| | | KL | |
| RRWRRWWRRWWRRWRR | 12 | MANLGYWLLALFVTMWTDVGLCK | 34 |
| | | KRPKP | |
| RGGRLSYSRRRFSTSTGR | 13 | LGTYTQDFNKFHTFPQTAIGVGAP | 35 |
| YGRKKRRQRRR | 14 | DPKGDPKGVTVTVTVTVTGKGDPX | 36 |
| | | PD | |
| RKKRRQRRR | 15 | PPPPPPPPPPPPPP | 37 |
| YARAAARQARA | 16 | VRLPPPVRLPPPVRLPPP | 38 |
| RRRRRRRR | 17 | PRPLPPPRPG | 39 |
| KKKKKKKK | 18 | SVRRRPRPPYLPRPRPPPFFPPRLPPR | 40 |
| | | IPP | |
| GWTLNSAGYLLGKINLKALAALA | 19 | TRSSRAGLQFPVGRVHRLLRK | 41 |
| KXIL | | | |
| LLILLRRRIRKQANAHSK | 20 | GIGKFLHSAKKFGKAFVGEIMNS | 42 |
| SRRHHCRSKAKRSRHH | 21 | KWKLFKKIEKVGQNIRDGIIKAGPA | 43 |
| | | VAVVGQATQIAK | |
| NRARRNRRRVR | 22 | ALWMTLLKKVLKAAAKAALNAVL | 44 |
| | | VGANA | |
| RQLRIAGRRLRGRSR | 23 | GIGAVLKVLTTGLPALISWIKRKRQ | 45 |
| | | Q | |
| KLIKGRTPIKFGK | 24 | INLKALAALAKKIL | 46 |
| RRIPNRRPRR | 25 | GFFALIPKIISSPLPKTLLSAVGSALG | 47 |
| | | GSGGQE | |
| KLALKLALKALKAALKLA | 26 | LAKWALKQGFAKLKS | 48 |
| KLAKLAKKLAKLAK | 27 | SMAQDIISTIGDLVKWIIQTVNXFTK | 49 |
| | | K | |
| GALFLGFLGAAGSTNGAWSQPKK | 28 | LLGDFFRKSKEKIGKEFKRIVQRIKQ | 50 |
| KRKV | | RIKDFLANLVPRTES | |
| KETWWETWWTEWSQPKKKRKV | 29 | PAWRKAFRWAWRMLKKAA | 51 |
| LKKLLKKLLKKLLKKLLKKL | 30 | KLKLKLKLKLKLKLKLKL | 52 |

C. Protein Delivery

In general, proteins are delivered to cells as a formulation that promotes entry of the proteins into a cell of interest. In a most basic form, lipid vehicles such as liposomes. For example, liposomes, which are artificially prepared vesicles made of lipid bilayers have been used to delivery a variety of drugs. Liposomes can be composed of naturally-derived phospholipids with mixed lipid chains (like egg phosphatidylethanolamine) or other surfactants. In particular, liposomes containing cationic or neutral lipids have been used in the formulation of drugs. Liposomes should not be confused with micelles and reverse micelles composed of monolayers, which also can be used for delivery.

A wide variety of commercial formulations for protein delivery are well known including PULSin™, Lipodin-Pro, Carry-MaxR, Pro-DeliverIN, PromoFectin, Pro-Ject, Chariot™ Protein Delivery reagent, BioPORTER™, and others.

Nanoparticles are generally considered to be particulate substances having a diameter of 100 nm or less. In contrast to liposomes, which are hollow, nanoparticles tend to be solid. Thus, the drug will be less entrapped and more either embedded in or coated on the nanoparticle. Nanoparticles can be made of metals including oxides, silica, polymers such as polymethyl methacrylate, and ceramics. Similarly, nanoshells are somewhat larger and encase the delivered substances with these same materials. Either nanoparticles or nanoshells permit sustained or controlled release of the peptide or mimetic, and can stabilize it to the effects of in vivo environment.

### IV. NUCLEIC ACID DELIVERY

As discussed above, in certain embodiments, expression cassettes are employed to express a transcription factor product, either for subsequent purification and delivery to a cell/subject, or for use directly in a genetic-based delivery approach. Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin | Banerji et al., 1983; Gilles et al., 1983; Grosschedl |
| Heavy Chain | et al., 1985; Atchinson et al., 1986, 1987; Imler et |
| | al., 1987; Weinberger et al., 1984; Kiledjian et al., |
| | 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; Redondo et |
| | al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II HLA- | Sherman et al., 1989 |
| DRa | |
| β-Actin | Kawamoto et al., 1988; Ng et al.; 1989 |
| Muscle Creatine | Jaynes et al., 1988; Horlick et al., 1989; Johnson |
| Kinase (MCK) | et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere and Tilghman et |
| | al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins | |
| (GRP94 and GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., 1986; |
| | Ondek et al., 1987; Kuhl et al., 1987; Schaffner et |
| | al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human | Muesing et al., 1987; Hauber et al., 1988; |
| Immunodeficiency | Jakobovits et al., 1988; Feng et al., 1988; Takebe et |
| Virus | al., 1988; Rosen et al., 1988; Berkhout et al., 1989; |
| | Laspia et al., 1989; Sharp et al., 1989; Braddock et |
| | al., 1989 |
| Cytomegalovirus | Weber et al., 1984; Boshart et al., 1985; Foecking |
| (CMV) | et al., 1986 |
| Gibbon Ape | Holbrook et al., 1987; Quinn et al., 1989 |
| Leukemia Virus | |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammary tumor | | al., 1981; Majors et al., |
| virus) | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Of particular interest are fibroblast specific promoters, such as Fibroblast-Specific Protein 1 (FSP1) promoter (Okada et al., 1998); collagen 1A1 (COL1A1) promoter (Hitraya et al., 1998) and Periostin (Postn) promoter (Joseph et al., 2008). Other promoters include muscle specific promoters and cardiac specific promoters such as the myosin light chain-2 promoter (Franz et al., 1994; Kelly et al., 1995), the a-actin promoter (Moss et al., 1996), the troponin 1 promoter (Bhaysar et al., 1996); the Na⁺/Ca²⁺ exchanger promoter (Barnes et al., 1997), the dystrophin promoter (Kimura et al., 1997), the α7 integrin promoter (Ziober and Kramer, 1996), the brain natriuretic peptide promoter (LaPointe et al., 1996), the αB-crystallin/small heat shock protein promoter (Gopal-Srivastava, 1995), a-myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the disclosure, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Multigene Constructs and IRES

In certain embodiments of the disclosure, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

C. Delivery of Expression Vectors

There are a number of ways in which expression vectors may introduced into cells. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adeno-associated virus (AAV) expression vector. AAV can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

Another expression vector may comprise a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Other viral vectors may be employed as expression constructs in the present disclosure. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the disclosure, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the disclosure for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment of the disclosure, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000™ is widely used and commercially available.

In certain embodiments of the disclosure, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

### V. METHODS OF TREATING MYOCARDIAL INFARCTION

As discussed above, the present disclosure provides for new post-MI therapies. In one embodiment of the present disclosure, methods for the treatment of subjects following an MI provides for one or more of the following outcomes as compared to an untreated patient: increased exercise capacity, increased blood ejection volume, decreased left ventricular end diastolic pressure, decreased pulmonary capillary wedge pressure, increased cardiac output, improved cardiac index, decreased pulmonary artery pressures, decreased left ventricular end systolic and diastolic dimensions, and decreased left ventricular wall stress, decreased wall tension and decreased wall thickness-same for right ventricle. In addition, the treatment may prevent progression to cardiac hypertrophy and ultimately heart failure.

Treatment regimens would vary depending on the clinical situation. However, in general, the treatment would begin at a time following an MI when the patient has been stabilized, but before significant cardiac fibroblast mobilization and scarring has begun. The patient may or may not be undergoing one or more other therapies for either prevention or treatment of an MI, or prevention or treatment of MI-related sequelae. This would mean initiating a treatment within about 24, 36, 48, 72, 96 hours of an MI, or within about 5, 6, 7, 8, 9 or 10 days of an MI. The therapy may continue for as long as cardiac fibroblasts would be active within the ischemic zone, such as up to 7 days, 14 days 21 days, 28 days, 1 month, 2 months, 3 months or longer.

A. Combined Therapies

In another embodiment, it is envisioned to use the transcription therapy inhibitor of the present disclosure in combination with other MI and post-MI therapeutic modalities, such as those discussed above. Combinations may be achieved by contacting cardiac cells/patients with a single composition or pharmacological formulation that includes both agents, or by contacting the cell with two distinct compositions or formulations, at the same time, wherein one composition includes the expression construct and the other includes the agent. Alternatively, the therapy using transcription factors may precede or follow administration of the other agent(s) by intervals ranging from minutes to weeks. In embodiments where the other agent and transcription factors are applied separately to the cardiac cells/patient, one would generally ensure that a significant period of time did not expire between the time of each delivery, such that the agent and transcription factors would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either transcription factors, or the other agent will be desired. In this regard, various combinations may be employed. By way of illustration, where the transcription factors are “A” and the other agent is “B,” the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated. One particular combination therapy involves anti-inflammatory agents, such as steroids or NSAIDs. Other traditional cardiac therapies are discussed below, and may also be usefully combined with the transcription factors discussed above.

B. Standard MI Therapeutic Intervention

Therapies for acute myocardial infarction are designed to restore perfusion as soon as possible to rescue the infracted myocardium. This is typically done by pharmaceutical intervention or by mechanical means, such as percutaneous coronary intervention (PCI) or coronary artery bypass grafting. Recent studies suggest that these treatments are more effective if the following guidelines are followed: <90 min for PCI and <30 min for lytics. Treatments outside these windows were associated with increased mortality and significantly increased risk of readmission for acute myocardial infarction or heart failure.

1. Drug Therapies

Thrombolytic therapy improves survival rates in patients with acute myocardial infarction if administered in a timely fashion in the appropriate group of patients. If PCI capability is not available within 90 minutes, then choice is to administer thrombolytics within 12 hours of onset of symptoms in patients with ST-segment elevation greater than 0.1 mV in 2 or more contiguous ECG leads, new left bundle-branch block (LBBB), or anterior ST depression consistent with posterior infarction. Tissue plasminogen activator (t-PA) is preferred over streptokinase as achieving a higher rate of coronary artery patency; however, the key lies in speed of the delivery.

Aspirin has been shown to decrease mortality and re-infarction rates after myocardial infarction. Again, delivery should be immediate, which should be chewed if possible. The treatment should continues indefinitely in the absence of obvious contraindication, such as a bleeding tendency or an allergy. Clopidogrel may be used as an alternative in cases of a resistance or allergy to aspirin (dose of 300 mg), but a higher dose of clopidogrel may have added benefit.

Platelet glycoprotein (GP) IIb/IIIa-receptor antagonist is another therapy in patients with continuing ischemia or with other high-risk features and to patients in whom a percutaneous coronary intervention (PCI) is planned. Eptifibatide and tirofiban are approved for this use, and abciximab also can be used for 12-24 hours in patients with unstable angina or NSTEMI in whom a PCI is planned within the next 24 hours.

Heparin and other anticoagulant agents have an established role as adjunct agents in patients receiving t-PA, but not in patients receiving streptokinase. Heparin is also indicated in patients undergoing primary angioplasty. Low molecular-weight heparins (LMWHs) have been shown to be superior to UFHs in patients with unstable angina or NSTEMI. Bivalirudin, a direct thrombin inhibitor, has shown promise in STEMI if combined with high-dose clopidogrel.

Nitrates have no apparent impact on mortality rate in patients with ischemic syndromes, but they are useful in symptomatic relief and preload reduction, so much so that all patients with acute myocardial infarction are given nitrates within the first 48 hours of presentation, unless contraindicated (i.e., in RV infarction). Beta-blockers may reduce the rates of reinfarction and recurrent ischemia, and thus are administered to patients with MIs unless a contraindication is present.

ACE inhibitors reduce mortality rates after myocardial infarction and thus are administered as soon as possible as long as no contraindications are and the patient remains stable. ACE inhibitors have the greatest benefit in patients with ventricular dysfunction. Continue ACE inhibitors indefinitely after myocardial infarction. Angiotensin-receptor blockers may be used as an alternative in patients who develop adverse effects, such as a persistent cough, although initial trials need to be confirmed.

2. PCI and Other Surgical Intervention

PCI is the treatment of choice in most patients with STEMI, assuming a door to balloon time of less than 90 minutes. PCI provides greater coronary patency (>96% thrombolysis), lower risk of bleeding, and instant knowledge about the extent of the underlying disease. Studies have shown that primary PCI has a mortality benefit over thrombolytic therapy. The choice of primary PCI should be individualized to each patient's presentation and timing. Primary PCI is also the treatment of choice in patients with cardiogenic shock, patients in whom thrombolysis failed, and those with high risk of bleeding or contraindications to thrombolytic therapy.

Emergent or urgent coronary artery graft bypass surgery is indicated in patients in whom angioplasty fails and in patients who develop mechanical complications such as a VSD, LV, or papillary muscle rupture.

C. Pharmacological Therapeutic Agents

Pharmacological therapeutic agents and methods of administration, dosages, etc., are well known to those of skill in the art (see for example, the “Physicians Desk Reference”, Klaassen's “The Pharmacological Basis of Therapeutics”, “Remington's Pharmaceutical Sciences”, and “The Merck Index, Eleventh Edition”, incorporated herein by reference in relevant parts), and may be combined with the disclosure in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

In addition to the transcription factors of the present disclosure, it should be noted that any of the following may be used to develop new therapeutic regimens in combination with the transcription factors.

1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present disclosure, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8, 11, 14, 17-eicosapentaenoic acid, eritadenine, furazabol, meglutol, melinamide, mytatrienediol, ornithine, γ-oryzanol, pantethine, pentaerythritol tetraacetate, a-phenylbutyramide, pirozadil, probucol (lorelco), β-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemmorage or an increased likelyhood of hemmoraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a B-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a B-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimefylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

g. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

7. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine administration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticmafen and urea.

c. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

d. Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

D. Surgical Therapeutic Agents

In certain aspects, the secondary therapeutic agent may comprise a surgery of some type, such as PCI. Surgery, and in particular a curative surgery, may be used in conjunction with other therapies, such as the present disclosure and one or more other pharmacologic agents. Such surgical approaches for vascular and cardiovascular diseases and disorders are well known to those of skill in the art, and are described elsewhere in this document.

E. Drug Formulations and Routes for Administration to Patients

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VI. EXAMPLES

The following examples are included to further illustrate various aspects of the disclosure. It should be appreciated by those of skill in the art that the techniques disclosed in the examples which follow represent techniques and/or compositions discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Animals. Animal work described in this study has been approved and conducted under the oversight of the University of Texas Southwestern Institutional Animal Care and Use Committee.

Isolation of mouse tail-tip fibroblast. For isolation of adult mouse tail-tip fibroblasts, tails were cut from 8-12 weeks old adult wild-type or αMHC-GFP mouse and were minced into 1-cm pieces with razor blades after peeling off the superficial dermis. The minced tails were placed in fibroblast growth medium (DMEM supplemented with 10% FBS and 1% (vol/vol) penicillin/streptomycin). Tail-tip fibroblasts migrated out from the explants within 1 week and were passaged one time before use.

Construction of human retroviral ORFs library. Gateway-compatible Human ORFs pEntry vectors were purchased from Thermo Fisher Scientific. Gateway-compatible retroviral destination vector, pMXs-gw, was a gift from Shinya Yamanaka (Addgene plasmid #18656) (Takahashi, 2006). The inventors transferred each ORF individually into pMXs-gw by performing site-specific LR recombination using Gateway LR Clonase II Enzyme Mix kit (Thermo Fisher Scientific).

Retroviruses production, cellular reprogramming and treatment. For retroviruses production, Platinum E cells were seeded into culture dishes (1×10⁵ cells/cm₂) one day before transfection in DMEM supplemented with 10% FBS and 1% penicillin/streptomycin. Cells reached ˜80% confluency on the day of transfection. DNA plasmids were transfected into Platinum E cells using FuGENE 6 transfection reagent. Twenty-four hours after transfection, wild type or αMHC-GFP tail-tip fibroblasts were seeded into culture dishes or plates that were precoated with SureCoat (Cellutron) for one hour at a density of 6×10⁴/cm². Forty-eight hours after transfection, polybrene was added to viral medium that was filtered through a 0.45-μm filter at a concentration of 8 μg/μL. The mixture replaced the growth medium in the cell culture plate with tail-tip fibroblasts. The viral infection was serially repeated twice. Twenty-four hours after the second infection, the viral medium was replaced with induction medium, composed of DMEM/199 (4:1), 10% conditioned medium obtained from neonatal rat cardiomyocyte culture, 10% FBS, 5% horse serum, 1% penicillin/streptomycin, 1% nonessential amino acids, 1% essential amino acids, 1% B-27, 1% insulin-selenium-transferrin, 1% vitamin mixture, and 1% sodium pyruvate (Invitrogen). Small molecule treatments were used throughout the reprogramming process 1004 dexamethasone (Dex), and 1004 nabumetone (Sigma).

Flow Cytometry. For flow Cytometry, cells were trypsinized and fixed with fixation buffer (BD Bioscience) for 15 min on ice. Fixed cells were washed with Perm/Wash buffer (BD Bioscience) for three times. Washed cells were incubated with mouse monoclonal anti-cardiac Troponin T (cTnT) antibody (Thermo Scientific) at 1:200 dilution and Rabbit anti-GFP antibody (Thermo Scientific) at 1:200 dilution in Perm/Wash buffer for 1 hr on ice. Cells then were washed with Perm/Wash buffer for three times followed by incubation with donkey anti-mouse Alexa fluor 647 (Invitrogen) at 1:200 and goat anti-rabbit Alexa fluor 488 at 1:200 (Invitrogen). Cells were washed with Perm/Wash buffer, and then analyzed using FACS Caliber (BD Sciences) and FlowJo software.

Immunocytochemistry. For immunocytochemistry, cells were fixed in 4% paraformaldehyde for 15 min and permeabilized with 0.1% Triton-X in room temperature. Cells were washed with PBS for three times followed by blocking with 10% goat serum for 1 hr. Cells then were incubated with mouse monoclonal anti-cardiac Troponin T (cTnT) antibody (Thermo Scientific) at 1:500 dilutions and rabbit anti-GFP antibody (Thermo Scientific) at 1:500 dilutions in 5% Goat serum for 1 hr. After washing with PBS three times, Cells then were incubated with donkey anti-mouse alexa fluor 647 (Invitrogen) at 1:500 and goat anti-rabbit Alexa fluor 488 at 1:500 (Invitrogen).

Quantitative mRNA measurement. Total RNA was extracted using TRIzol (Invitrogen) according vender's protocol. RNAs were retrotranscribed to cDNA using iScript Supermix (Bio-Rad). qPCR was performed using Kapa Sybr Fast (Kapa Biosystems). mRNA levels were normalized by comparison to Gapdh mRNA.

Immunoprecipitation and Western Blot Analysis. Flag-epitope tag ZNF281 fusion protein were co-expressed with myc-epitope tag fusion GATA4, HAND2, MEF2C or TBX5 in HEK293 cells for 48 hours. Cell lysates were incubated overnight with 1 μg of mouse monoclonal anti-FLAG antibody (Sigma). The cell lysates were pulldown using magnetic Protein G Dynabeads (Invitrogen) and then the Flag-ZNF281 was eluted using 0.5 mg/ml of free Flag peptide (Sigma). The final elution and the input obtained before the immunoprecipitation was analyzed by SDS-PAGE western blot using a mouse monoclonal anti-Myc antibody (Novex) or mouse monoclonal anti-FLAG antibody (Sigma).

RNA-seq and Gene Ontology Analysis. For RNA-seq, total RNA was extracted from TTFs transduced with indicated retroviruses using TRIzol (Invitrogen) according vender's protocol. Illumina RNA-seq was performed by the University of Texas Southwestern Microarray Core Facility. RNA-Seq and Transcriptome Analysis were performed as described in (Zhou et al., 2015). Briefly, quality assessment of the RNA-seq data was done using NGS-QC-Toolkit. Reads with more than 30% nucleotide with phred quality scores less than 20 were removed from further analysis. Quality filtered reads were then aligned to the mouse reference genome GRCm38 (mm10) using the Hisat (v 2.0.0) aligner using default setting. Aligned reads were counted using featureCounts (v1.4.6) per gene ID. Differential gene expression analysis was done using the R package edgeR (v 3.8.6). For each comparison, genes were required to have 10 cpm (counts per million) in at least 3 samples to be considered as expressed. They were used for normalization factor calculation. Gene differential expression analysis was done using GLM approach following edgeR official documentation. Cutoff values of -fold change greater than 2 and FDR<0.01 were then used to select for differentially expressed genes between sample group comparisons. The DAVID gene functional annotation and classification tool was used to annotate the list of differentially expressed genes with respective Gene Ontology (GO) terms and perform GO enrichment analysis for molecular and biological functional categories.

ChIP-seq and Gene Ontology and Pathway Analysis. For Chip-seq, TTFs were transduced with indicated retroviruses. Two days after retroviral transduction, TTFs were crosslinked with 1% formaldehyde in PBS for 30 min and neutralized by the addition of glycine to a final concentration of 0.125 M for 5 min. TTFs were harvested and washed with cold PBS, then perform ChIP using indicating antibodies and ChIP-IT® Express Chromatin Immunoprecipitation Kits (Active Motif) according to according vender's protocol. Subsequent library construction and massive parallel sequencing will be performed at UTSW Genomics and microarray core facility. For ChIP-Seq analysis, raw reads are mapped to GRCm38 (mm10) using bowtie2 (ver2.2.8). An average of ˜50 million uniquely mapped (single end) reads were obtained per sample. Peak calling was performed using MACS2 (ver2.1.0). Resulting peak files were processing using bedTools (ver2.26) and deepTools (Ramirez et al., 2016) to generate coverage heatmaps and obtain overlapping regions. The Genomic Regions Enrichment of Annotations Tool kits (McLean et al., 2010) were used for Gene Ontology and Pathway Analysis

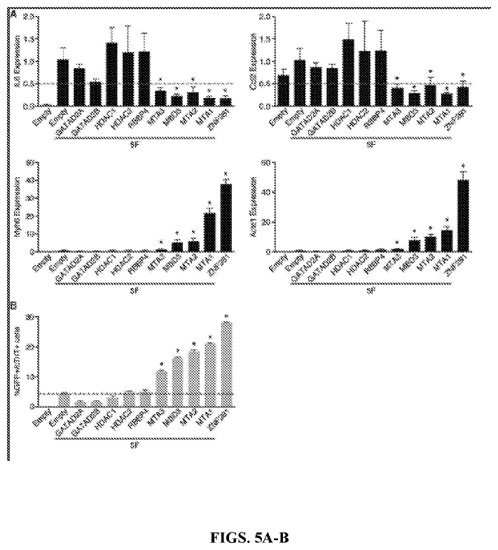

Statistical Analyses. All data are presented as mean with SEM and have n=3 per group (except FIG. 5B, with n=6 per group). P values were calculated with either unpaired/two-way t-test or one-way ANOVA. Statistical analyses were run using the GraphPad Prism 7 software package (GraphPad Software). P<0.05 was considered significant in all cases after corrections were made for multiple pairwise comparisons.

### Example 2—Results

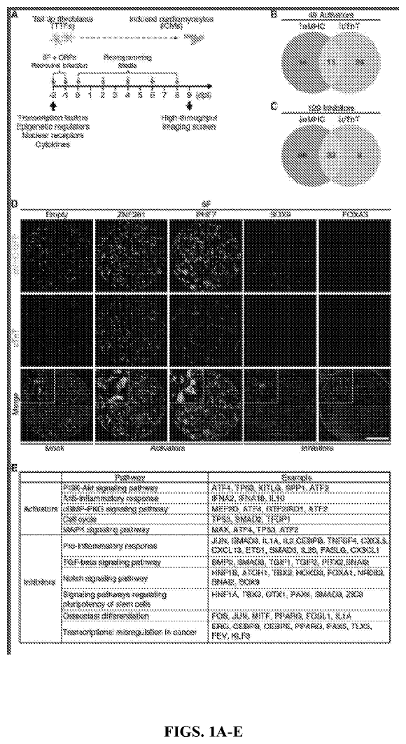

Identification of activators and inhibitors of cardiac reprogramming in adult fibroblasts. The inventors' previously optimized cardiac reprogramming protocol with five factors (AGHMT, also called 5F) was able to reprogram ˜3% efficiency of adult TTFs to iCMs as measured by activation of a cardiac-specific αMHC-GFP transgene and cardiac troponin T (cTnT) immunostaining (Zhou et al., 2015). To identify additional regulators of cardiac reprogramming, the inventors created a retroviral expression library consisting of 1,052 open reading frame (ORF) cDNAs representing 786 human transcription factor, cytokine, epigenetic regulator and nuclear receptor genes (Table 51). The inventors screened this expression library for activators and inhibitors of cardiac reprogramming by expressing individual cDNAs together with 5F in isolated TTFs from αMHC-GFP mice, as schematized in FIG. 1A. After 9 days, a high-throughput cell analyzer system was used to image and quantify cardiac reprogramming based on αMHC-GFP and cTnT expression. Activators were defined as genes that increased αMHC-GFP or cTnT expression with a Z-score ≥2, whereas genes with Z-scores ≤−2 for αMHC-GFP or cTnT expression were defined as inhibitors. This screen led to the discovery of 49 potential activators and 129 potential inhibitors of cardiac reprogramming (FIGS. 1B-C; Table S2).

Among the 49 activators, 25 enhanced αMHC-GFP expression; 35 enhanced cTnT expression; and 11 enhanced expression of both cardiac markers (FIG. 1B). The two strongest activators were PHD finger protein 7 (PHF7), a histone H3 binding protein expressed only in the male germ line (Yang et al., 2012); and Zinc finger protein 281 (ZNF281), a zinc finger transcription factor about which little is known (FIG. 1D, Table S2). Among the 129 inhibitors, 121 inhibited αMHC-GFP expression; 41 inhibited cTnT expression; and 33 inhibited both cardiac markers (FIG. 1C). Some of the repressors, such as forkhead box protein A3 (FOXA3), practically abolished 5F-mediated cardiac reprogramming (FIG. 1D and Table S2). Cell numbers were unaffected by the inhibitors, suggesting that they acted directly on the reprogramming process rather than through indirect mechanisms, such as causing cell death.

To identify key pathways that regulate cardiac reprogramming, the inventors performed pathway enrichment analysis for activator and inhibitor genes. Given that this was a genome-wide screen, they expected that this analysis would identify pathways known to regulate cardiac reprogramming. Indeed, the PI3K-AKT signaling pathway, which has been shown to enhance cardiac reprogramming (Zhou et al., 2015), was among the most enriched pathways associated with the activators. Other enriched pathways associated with the activators were the anti-inflammatory pathway, the cGMP-PKG signaling pathway, the cell cycle pathway and MAPK signaling pathway (FIG. 1E). It is noteworthy that the TGF-β and Notch signaling pathways, which have been shown to negatively regulate cardiac reprogramming (Abad et al., 2017; Ifkovits et al., 2014), were among the most enriched pathways associated with the inhibitors. Other enriched pathways associated with the inhibitors were the pro-inflammatory pathway, signaling pathways regulating pluripotency of stem cells, osteoclast differentiation and transcriptional misregulation in cancer (FIG. 1E).

Since inflammatory signaling pathways were associated with both activators and inhibitors, the inventors examined the functions of each individual gene within these pathways. Interestingly, they found that most of the identified activators possessed anti-inflammatory functions, including several anti-inflammatory cytokines, such as IFNA2, IFNA16 and IL10. Consistent with these findings, they found that most identified inhibitors were pro-inflammatory, including several pro-inflammatory cytokines, such as IL1A, IL2 and IL26, and the inflammatory response transcription factor CEBβ (FIG. 1E).

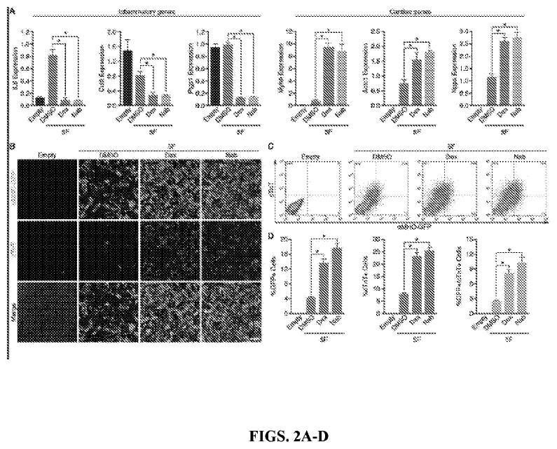

Anti-inflammatory drugs promote cardiac reprogramming. Given that activators and inhibitors were associated with opposing inflammatory functions, the inventors postulated that inhibition of the inflammatory response by anti-inflammatory drugs would also enhance reprogramming, so the inventors tested the effects of two anti-inflammatory drugs, dexamethasone (Dex), a steroidal anti-inflammatory drug, and the cyclooxygenase enzyme inhibitor nabumetone (Nab), a non-steroidal anti-inflammatory drug, on the reprogramming process. Anti-inflammatory drugs (10 μM) were added to 5F-reprogrammed TTFs post-viral infection. After 7 days of drug treatment, the inventors harvested RNA and examined the transcript levels of inflammatory (IL6, Ccl2, and Ptgs1) and cardiac markers (Myh6, Actc1 and Nppa) by q-PCR. Addition of anti-inflammatory drugs to 5F decreased expression of inflammatory markers, as expected, but increased the expression of cardiac markers from 2- to 10-fold, indicating enhanced reprogramming efficiency (FIG. 2A).

To further confirm the cardiogenic influence of anti-inflammatory drugs on the reprogramming process, the inventors performed immunocytochemistry (FIG. 2B) and flow cytometry (FIGS. 2C-D) for the cardiac markers αMHC-GFP and cTnT, respectively. Both assays confirmed the capacity of anti-inflammatory drugs to promote cardiac reprogramming.

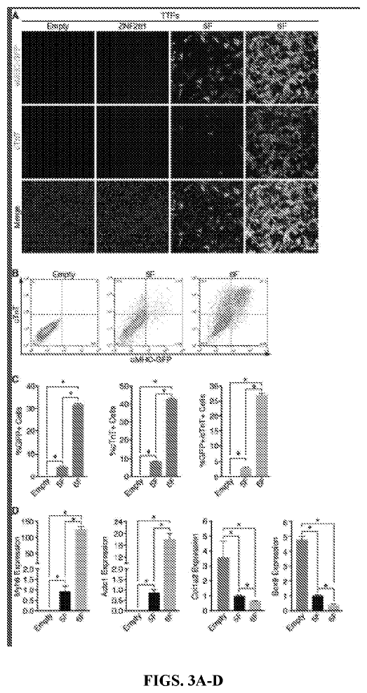

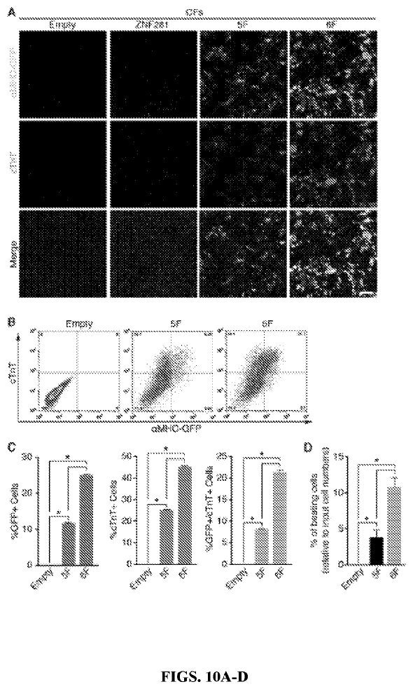

ZNF281 enhances cardiac reprogramming of adult fibroblasts. PHF7 and ZNF281 were the two strongest activators identified from retroviral cDNA expression screen with 5F (FIG. 1D; Table S2). PHF7 is only expressed in the male germ line (Yang et al., 2012), whereas ZNF281 has a broad expression pattern with enriched expression in the heart (FIG. 51). The inventors therefore focused their initial attention on ZNF281 and explored its ability to enhance the activity of 5F. They refer to the reprogramming mix of 5F plus ZNF281 as 6F. The inventors validated the results of their screen by assessing GFP and cTnT expression in αMHC-GFP TTFs following 5F and 6F reprogramming after 7 days (FIG. 3A). Flow cytometry showed that addition of ZNF281 to 5F generated ˜33% αMHCGFP+, ˜45% cTnT+ and ˜28% αMHC-GFP+/cTnT+ TTFs after 7 days of reprogramming (FIGS. 3B-C). This TTF reprogramming efficiency using 6F is noteworthy when considering the relatively low statistical likelihood of each fibroblast taking up all five or six separate retroviruses encoding the reprogramming factors. The inventors also examined the expression of cardiac and fibroblast transcripts by q-PCR. Addition of ZNF281 to 5F increased the expression of cardiac marker genes, Myh6 and Actc1, by ˜120-fold and ˜20-fold, respectively, and decreased expression of fibroblast marker genes, Col1a2 and Sox9, by ˜30% and ˜60%, respectively (FIG. 3D). The inventors also validated some of these results using αMHC-GFP adult cardiac fibroblasts (CF). The addition of ZNF281 to 5F increased the expression of GFP and cTnT in αMHC-GFP CFs evaluated by immunocytochemistry after 7 days of reprogramming (FIG. 10A). This data was also corroborated by FACS analysis. The addition of ZNF281 to 5F generated ˜25% αMHC-GFP+, ˜45% cTnT+ and ˜21% αMHC-GFP+/cTnT+ iCMs. (FIG. 10B-C). Interestingly ZNF281 in presence of the 5F also induce a 2-fold increase in the number of beating cells after 4 weeks of reprogramming (FIG. 10D).

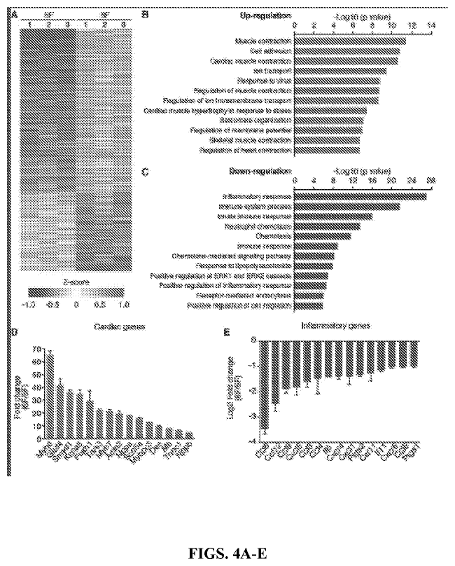

To further define the influence of ZNF281 on the reprogramming process, the inventors performed RNA-seq using adult TTFs reprogrammed for 7 days with 5F or 6F. Using a two-fold cutoff and FDR <0.01 threshold for inclusion, they identified 1,000 up-regulated genes and −500 down-regulated genes in 6F compared to 5F treated TTFs (FIGS. 4A-C). Gene ontology enrichment analysis revealed that ZNF281 selectively up-regulated genes associated with muscle contractility (FIGS. 4B and 4D), suggesting that ZNF281 globally enhanced cardiac reprogramming. Interestingly, the top gene ontology terms enriched in the genes that were down-regulated by ZNF281 were all related to the inflammatory response (FIGS. 4C and 4E), suggesting a dual role for ZNF281 in activation of cardiac and suppression of inflammatory gene programs.

Co-repression of inflammatory genes by ZNF281 and NuRD complex components. Previously, it was reported that ZNF281 associates with the Nucleosome Remodeling Deacetylase (NuRD) complex to repress transcription (Fidalgo et al., 2012; Fidalgo et al., 2016). The NuRD complex is an ATP-dependent chromatin remodeling complex that contains multiple subunits. Notably, the inventors identified the NuRD complex component MTA1 as an activator of cardiac reprogramming in their screen (Table S2). Because the NuRD complex has also been shown to repress inflammatory signaling (Ramirez-Carrozzi et al., 2006), the inventors tested other NuRD complex subunits including MTA2, MTA3, MBD3, GATAD2A, GATAD2B, RBBP4, HDAC1 and HDAC2 in the reprogramming assay with 5F in adult TTFs. Indeed, addition of four of the NuRD complex subunits (MTA1, MTA2, MTA3 and MBD3) to 5F substantially decreased expression of the inflammatory markers (IL6, Ccl2) and increased expression of cardiac markers (Myh6, Actc1) (FIG. 5A). Addition of NuRD complex subunits also promoted cardiac reprogramming, as measured by flow cytometry using the cardiac markers αMHC-GFP and cTnT (FIG. 5B, FIGS. S2A-B).

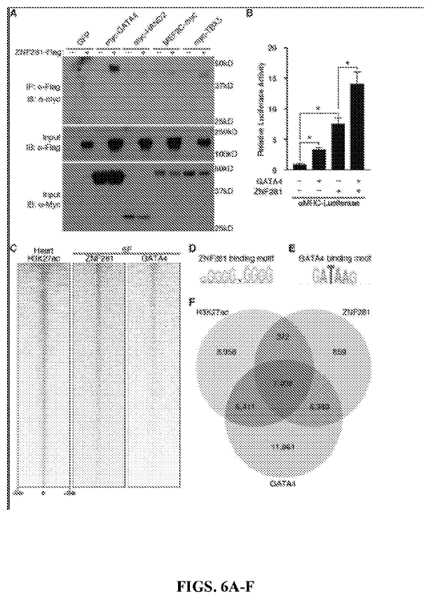

ZNF281 is a GATA4 coactivator. Previous reports described the influence of ZNF281 on pluripotency, stemness and epithelial-mesenchymal transition (EMT) (Hahn and Hermeking, 2014). However, the potential involvement of ZNF281 in cardiac development has not been previously explored. To begin to define the molecular mechanism whereby ZNF281 enhances cardiac reprogramming, the inventors performed co-immunoprecipitation assays with ZNF281 and each reprogramming transcription factor in transfected HEK293 cells. They found that FLAG-tagged ZNF281 co-immunoprecipitated with Myc-tagged GATA4 but not with the other three factors (FIG. 6A).

To determine if ZNF281 could directly activate cardiac genes, the inventors examined the ability of ZNF281 to activate a luciferase reporter controlled by the αMHC promoter (αMHC-Luciferase). Indeed, ZNF281 activated the αMHC-Luciferase reporter ˜7-fold and when ZNF281 was co-expressed with GATA4, αMHC-Luciferase was activated ˜15-fold (FIG. 6B), suggesting that ZNF281 and GATA4 synergize to activate the αMHC promoter. The finding that ZNF281 alone could activate the αMHC reporter (FIG. 3A), but could not activate the endogenous αMHC gene or other cardiac genes alone may suggest that it relies on GATA4 as a pioneer factor to open target sites on cardiac genes that are otherwise inaccessible in native chromatin.

ZNF281 broadly co-occupies cardiac enhancers with GATA4. To further understand the molecular relationship between ZNF281 and GATA4 in the cardiac reprogramming process, the inventors examined the genomic locations of ZNF281 and GATA4 at an early stage of the reprogramming process (2 days post-infection with 6F) by chromatin immunoprecipitation (ChIP) with antibodies to endogenous proteins followed by high-throughput sequencing (ChIP-seq) (FIGS. 6C-6F). The inventros performed de novo motif discovery on the binding peaks for ZNF281 and GATA4. The most significantly enriched motif associated with ZNF281 binding was GGGGTGGGG (FIG. 6D). For GATA4, the most enriched motif was GATAAG, which matches the consensus sequence for DNA binding of this transcription factor (FIG. 6E). ChIP-seq identified 14,623 peaks for ZNF281 and 30,664 peaks for GATA4 (FIG. 6F).

Based on the interaction of ZNF281 with GATA4 (FIG. 6A), the inventors predicted that ZNF281 co-occupies genomic sites with GATA4 on cardiac enhancers. Indeed, they found a high degree of overlap of ZNF281 and GATA4 genomic binding sites. Among the 14,623 peaks of ZNF281, the inventors found that 91.6% (13,392/14,623) overlapped with peaks of GATA4 binding, and only 8.4% of ZNF281 peaks did not overlap with GATA4, indicating the co-occupancy of ZNF281 and GATA4 on endogenous genomic sites at an early stage of the cardiac reprogramming process (FIG. 6F). In contrast, the overlapping peaks of ZNF281 and GATA4 only account for 43.7% of the total GATA4 peaks (13,392/30,664) (FIG. 6F), suggesting that GATA4 likely co-occupies additional sites with other reprogramming factors besides ZNF281.

To assess genome-wide localization of ZNF281 on cardiac enhancers, the inventors established a chromatin landscape for heart tissue using H3K27ac ChIP data from the Encode project (Mouse et al., 2012) to reveal cardiac enhancers. Rank ordering of all cardiac enhancers based on H3K27ac enrichment revealed that GATA4 bound to the majority, 57.1% (12,414/21,744), of active cardiac enhancers, consistent with the established role of GATA4 in heart development. ZNF281 bound to 33.9% (7,375/21,744) of active cardiac enhancers, while ZNF281 and GATA4 co-occupied with 32.2% (7,003/21,744) of cardiac enhancers (FIGS. 6C and 6F). Genome-wide binding data for ZNF281 and GATA4 suggested that ZNF281 is most commonly associated with GATA4 on cardiac enhancers, defined by the presence of H3K27ac (FIGS. 6C and 6F).

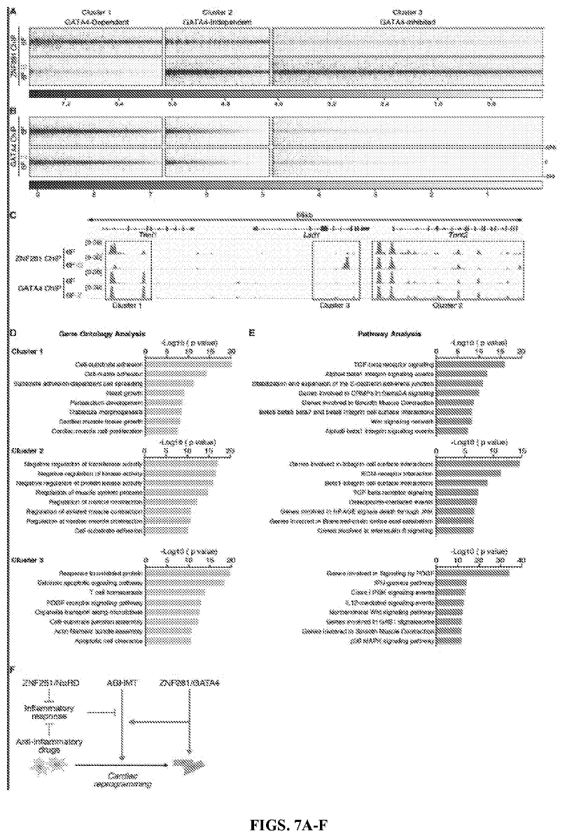

GATA4 recruits ZNF281 to cardiac enhancers. The inventors compared the occupancy patterns of ZNF281 in the presence (referred to as “6F”) or absence of GATA4 (referred to as “6F-G”) (FIG. 7A). Additionally, they examined the genomic occupancy pattern of GATA4 in the absence of ZNF281 (referred to as “6F-Z”) following reprogramming (FIG. 7B). The inventors found that the occupancy pattern of GATA4 throughout the genome was not dramatically affected by the presence or absence of ZNF281 (FIG. 7B). However, the presence of GATA4 strongly impacted the genomic occupancy pattern of ZNF281 (FIG. 7A). They assigned 33,934 ZNF281 binding peaks into three clusters based on how GATA4 influenced ZNF281 occupancy: a GATA4-dependent cluster (cluster 1, 8,882/33,934 peaks); a GATA4-independent cluster (cluster 2, 7,003/33,934 peaks); and a GATA4-inhibited cluster (cluster 3, 18,049/33,934 peaks) (FIGS. 7A-B). Interestingly cluster 3, in which the occupancy of ZNF281 was inhibited by the presence of GATA4, also correlated with lower binding affinity of GATA4 compared with clusters 1 and 2 (FIGS. 7A-B). It seems that the higher-affinity GATA4 binding sites in cluster 1 competed ZNF281 away from the lower affinity GATA4 binding sites in cluster 3. For example, a region which is located at chr1:137,694,960-137,749,970 (mm9) shows binding peaks that belong to three different clusters (FIG. 7C). Binding peaks that associated with the sarcomere genes Tnni1 and Tnnt2, which encode subunits of the troponin complex involved in regulating muscle contraction were assigned to cluster 1 and cluster 2, respectively. Binding peaks that associated with a non-muscle gene ladinin-1 (Lad1), which encodes a basement membrane protein, were assigned to cluster 3.

To investigate the functional significance of the GATA4 and ZNF281 binding peaks in the three clusters, the inventors performed gene ontology and pathway enrichment analysis using the Genomic Regions Enrichment of Annotations Tool (GREAT) (McLean et al., 2010). For gene ontology enrichment analysis, they found that most of the gene ontology terms enriched in clusters 1 and 2 were heart or muscle related (FIG. 7D), whereas most of the gene ontology terms enriched in cluster 3 were related to stress and inflammatory responses. For pathway enrichment analysis, several pathways, such as the TGF-beta and Wnt signaling pathways, which are known to be important for cardiogenesis, heart repair and cardiac reprogramming (Ifkovits et al., 2014), were enriched in clusters 1 and 2, whereas inflammatory response pathways were enriched in cluster 3 (FIG. 7E). The inventors conclude that GATA4 directly recruits ZNF281 to cardiac enhancers to activate cardiac gene expression.

In contrast to the effect on cardiac enhancers, GATA4 did not attenuate ZNF281 binding to inflammatory enhancers (FIGS. 7A, 7D and 7E). Additionally, ZNF281 served as a repressor instead of an activator of inflammatory genes, suggesting that ZNF281 acts through distinct mechanisms to regulate expression of cardiac and inflammatory genes.

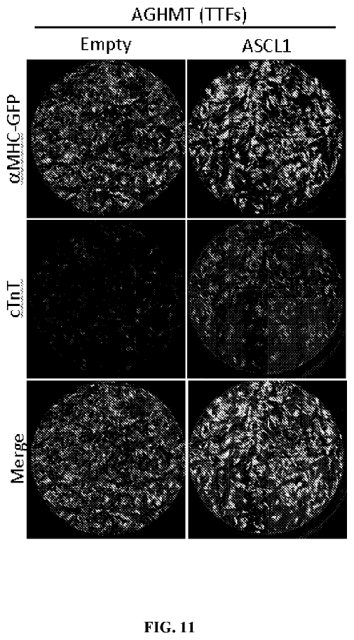

ASCL1 enhances cardiac reprogramming of adult fibroblasts. Our initial focus was on activators that have enriched expression in the heart. However, we found several activators that have little expression in the heart but still strongly enhanced cardiac reprogramming. For example, Achaete-scute homolog 1(ASCL1), a helix-loop-helix family of transcription factors expressed only in the brain greatly enhanced reprogramming (FIG. 11, Table S2). PHD finger protein 7 (PHF7), a histone H3 binding protein expressed only in the male germ line also enhanced reprogramming (FIG. 1D, Table S2).

### Example 3—Discussion

Here, the inventors performed an unbiased screen for regulators of adult cardiac reprogramming and identified 178 new activators and inhibitors that belong to various biological pathways. These different regulators revealed that anti-and pro-inflammatory factors evoke opposing effects on cardiac reprogramming. The inventors found that pro-inflammatory molecules prevented reprogramming, whereas anti-inflammatory drugs enhanced cardiac reprogramming. Among the identified activators, the zinc finger transcription factor ZNF281 showed the most potent stimulatory activity. The effect of ZNF281 on cardiac reprogramming appears to be mediated by association with GATA4 on cardiac enhancers and by inhibition of inflammatory signaling, which antagonizes cardiac reprogramming (FIG. 7F).

Stimulation of cardiac reprogramming by ZNF281. The inventors' unbiased screen identified ZNF281 as an activator of cardiac reprogramming. Previous reports described the influence of ZNF281 on pluripotency, stemness and EMT (Hahn and Hermeking, 2014). However, the importance of ZNF281 in cardiac development and cardiogenesis has not been previously recognized. The inventors show that ZNF281 functions as a positive regulator of cardiogenesis by associating with GATA4 on cardiac enhancers. Using the inventors' 6F cardiac reprogramming assay, they show that ZNF281 interacts with GATA4 to synergistically activate cardiac genes including those encoding cardiac transcription factors, calcium handling proteins, cardiac metabolic enzymes and components of the sarcomere. ZNF281 is expressed in a variety of tissues during various developmental stages. Homozygous deletion of ZNF281 in mice results in embryonic lethality between embryonic day 7.5 and 8.5, prior to the formation of primitive ventricles and atria (Fidalgo et al., 2011; Xin et al., 2013). The inventors speculate that ZNF281 may also have important functions in the early stage of cardiac development.

GATA4 as a potential pioneer factor for cardiac reprogramming. Little is known about how ectopic expression of cardiac reprogramming factors drives the conversion of fibroblasts to cardiomyocytes. It has been suggested that reprogramming requires pioneer factors to first engage and open target sites in chromatin and confer competency for other factors to bind (Soufi et al., 2015). Previously, for the albumin gene enhancer in liver precursor cells, it was shown that GATA4 serves as a pioneer factor and initiates chromatin opening (Cirillo et al., 2002). Cardiac gene expression during heart development requires regulated interactions among the transcription factors, GATA4, TBX5, and NKX2-5. The combined cooperativity of these factors is critical for the expansion of cardiac progenitors and for the regulation of the cardiac differentiation program (Luna-Zurita et al., 2016). Moreover, it has been shown in human cardiomyocytes that GATA4 broadly co-occupies cardiac enhancers with TBX5 (Ang et al., 2016). A human GATA4 mutant that disrupts TBX5 association led to aberrant chromatin states and cellular dysfunction, including those related to morphogenetic defects which induced impaired contractility, calcium handling, and metabolic activity (Ang et al., 2016).

The inventors show that ZNF281 does not bind to cardiac enhancers in fibroblasts without the presence of GATA4, indicating that ZNF281 is unlikely to be a pioneer factor that opens chromatin. However, they show that ZNF281 requires the presence of the pioneer factor GATA4 to bind cardiac enhancers. Understanding the mechanism by which pioneer factors and other transcription factors interact and induce conformational changes in chromatin structure will be necessary to decipher the molecular mechanism involved in cardiac reprogramming.

The influence of inflammation on cardiac reprogramming. Following injury, the inflammatory infiltration is among the earliest responses, which is necessary to clear cellular debris and promote scar formation (Aurora and Olson, 2014a). Recent studies revealed both positive and negative roles for the inflammatory response in cardiac repair and regeneration. Inhibition of C/EBP signaling in adult epicardium reduces injury-induced neutrophil influx, leading to a reduction of fibrosis and maintenance of cardiac function (Huang et al., 2012). In contrast, the injury-induced macrophage response is necessary for heart regeneration in neonatal and zebrafish (Aurora et al., 2014b; Lai et al., 2017). These results show that the inflammatory response acts as a barrier for cardiac reprogramming. In contrast, it has been reported that efficient iPSC reprogramming depends on activation of the inflammatory response to enhance chromatin remodeling that favors iPSC formation by toll-like receptor 3 mediated repression of the expression of epigenetic modifiers (Lee et al., 2012). The opposing effects of inflammation on cardiac versus iPSC reprogramming suggest that these two types of reprogramming employ distinct regulatory mechanisms. Given that embryonic stem cells are pluripotent and able to generate different cell types, whereas cardiomyocytes are terminally differentiated cells, it is possible that inflammation promotes epigenetic remodeling that is favorable for iPSC but not for cardiac reprogramming.

Treatment of 5F reprogrammed adult TTFs with anti-inflammatory drugs, dexamethasone and nabumetone, greatly enhanced cardiac reprogramming efficiency. Multiple stimuli can trigger the inflammatory response in cardiac reprogramming. Viral infection used for delivery of the reprogramming factors is one source of the inflammatory response. GATA4, one of the reprogramming factors, has also been shown to induce inflammation in fibroblasts (Kang et al., 2015). It is interesting to note, in this regard, that inflammation and inflammatory cell filtration are hallmarks of MI and reperfusion injury (Aurora and Olson, 2014a). Ischemic cardiac injury triggers inflammatory reactions accompanied by cytokine release and inflammatory cell filtration into the infarct region.

Influence of the NuRD complex on cardiac reprogramming. The chromatin remodeling complex NuRD is a well-known transcriptional repressor and plays an important role in various cellular processes. For example, NuRD has been shown to repress the pro-inflammatory gene expression in lipopolysaccharide stimulated macrophage (Ramirez-Carrozzi et al., 2006). The inventors show that in addition to direct cardiac gene activation, ZNF281 and some components of the NuRD complex also represses inflammatory signaling to activate cardiac reprogramming. Unlike cardiac reprogramming, ZNF281 has been shown to repress iPSC reprogramming through NuRD complex-mediated Nanog and other embryonic stem cell specific gene repression (Fidalgo et al., 2011). Given that the inflammatory response is critical for iPSC formation (Lee et al., 2012), the inventors surmise that in addition to repression of embryonic stem cell specific genes, the anti-inflammatory effect of the ZNF281/NuRD complex also contributes to the repression of iPSC reprogramming. They cannot exclude the possibility that the NuRD complex has a more direct role on activation of the cardiac gene program. With this regard, preservation of the identity of heart or skeletal muscle cell types has also been shown to depend on NuRD mediated epigenetic repression of alternate lineage gene expression. Loss of the NuRD subunit in the heart triggers aberrant expression of skeletal muscle gene expression and leads to hybrid striated muscle tissue (Gomez-Del Arco et al., 2016).

Clinical implications. Direct reprogramming of adult fibroblasts to cardiomyocytes represents a potential approach for repairing the heart following injury. As an activator of cardiac reprogramming, ZNF281 offers new insight into the molecular basis of this process. Additionally, the inventors' finding that ZNF281 represses the inflammatory response reveals a role for anti-inflammatory signaling in cardiac reprogramming and highlights the potential clinical application of commonly used anti-inflammatory drugs in cardiac repair.

TABLE S1
Genes included in human retroviral ORF cDNA library.
Gene Symbol

| | | | | | |
|---|---|---|---|---|---|
| AATF | ADIPOQ | ADNP | AEBP1 | AFF4 | AHR |
| AIMP1 | ALS2CR8 | ALX1 | ARGFX | ARID3A | ARNT |
| ARNT2 | ASCL1 | ASCL2 | ASXL1 | ATAD3B | ATF2 |
| ATF4 | ATF5 | ATF6 | ATF6B | ATF7 | ATOH1 |
| AURKA | AURKB | AURKC | BARX1 | BATF | BATF2 |
| BATF3 | BCL6 | BLZF1 | BMP1 | BMP10 | BMP2 |
| BMP3 | BMP5 | BMP7 | BMP8A | BRPF1 | BRPF3 |
| BUD31 | C17ORF77 | C1QTNF4 | C21ORF66 | CBFA2T2 | CBFA2T3 |
| CBFB | CBL | CBX5 | CBX6 | CBX7 | CBX8 |
| CCDC104 | CCDC69 | CCL1 | CCL11 | CCL13 | CCL14 |
| CCL15 | CCL17 | CCL18 | CCL2 | CCL21 | CCL22 |
| CCL23 | CCL24 | CCL25 | CCL26 | CCL27 | CCL28 |
| CCL3 | CCL3L3 | CCL4 | CCL4L1 | CCL5 | CCL7 |
| CCL8 | CD2 | CD200R1 | CD40LG | CD99P1 | CDYL |
| CDYL2 | CEBPB | CEBPD | CEBPE | CEBPG | CERS1 |
| CERS2 | CERS4 | CFL2 | CHIA | CIR1 | CITED1 |
| CITED2 | CKLF | CLCF1 | CMTM1 | CMTM2 | CMTM3 |
| CMTM4 | CMTM5 | CMTM6 | CMTM7 | CMTM8 | CNBP |
| CNOT7 | CNTF | CNTNAP3 | COBLL1 | CPA3 | CPB2 |
| CREB1 | CREB3 | CREB3L2 | CREB3L4 | CREB5 | CREBL2 |
| CREBRF | CREM | CRHR2 | CRLF1 | CRX | CSDA |
| CSF1 | CSF2 | CSF3 | CSRNP3 | CSRP2BP | CTBP1 |
| CTBP2 | CTCF | CTF1 | CTNNB1 | CUX1 | CX3CL1 |
| CXCL10 | CXCL11 | CXCL12 | CXCL13 | CXCL14 | CXCL16 |
| CXCL3 | CXCL5 | CXCL6 | CXCL9 | DBT | DLX1 |
| DLX3 | DLX4 | DLX6 | DMBX1 | DMRT1 | DPPA4 |
| DRAP1 | DRGX | E2F2 | E2F3 | E2F6 | E2F7 |
| E2F8 | E4F1 | EBF1 | EBI3 | EDF1 | EDN1 |
| EGR2 | EHF | ELF1 | ELF2 | ELF4 | ELF5 |
| ELK1 | ELK3 | ELK4 | EMB | END1 | ERF |
| ERG | ESAM | ESR1 | ESR2 | ESRRA | ETS1 |
| ETS2 | ETV3 | ETV7 | FAM3B | FAM3D | FASLG |
| FBLN1 | FEV | FL1 | FLI1 | FLT3LG | FOS |
| FOSB | FOSL1 | FOSL2 | FOXA1 | FOXA2 | FOXA3 |
| FOXD4 | FOXD4L6 | FOXJ1 | FOXJ2 | FOXL1 | FOXL2 |
| FOXM1 | FOXN3 | FOXO3 | FOXP1 | FOXP3 | FOXP4 |
| FUBP1 | GABPA | GABPB1 | GAS7 | GATA1 | GATA2 |
| GATA3 | GATA5 | GATAD1 | GATAD2A | GATAD2B | GBX2 |
| GDF10 | GDF15 | GDF2 | GDF3 | GDF6 | GIN1 |
| GLIS3 | GMEB1 | GPBP1 | GPER | GPI | GREM1 |
| GREM2 | GRN | GTF2H2 | GTF2H2C | GTF2H3 | GTF2H4 |
| GTF2I | GTF2IRD1 | HAT1 | HBM | HCFC1 | HDAC1 |
| HDAC3 | HDAC4 | HDAC7 | HDAC8 | HES1 | HES6 |
| HESX1 | HEYL | HHEX | HIF3A | HINFP | HIRA |
| HLF | HLX | HLX1 | HMG20B | HMGA1 | HMGB |
| HMGB1 | HMGB2 | HNF1A | HNF1B | HNF4A | HNRNPAB |
| HOPX | HOXA1 | HOXA11 | HOXA5 | HOXA6 | HOXA7 |
| HOXB1 | HOXB13 | HOXB5 | HOXB6 | HOXB7 | HOXB9 |
| HOXC4 | HOXC5 | HOXC6 | HOXC8 | HOXD1 | HOXD10 |
| HOXD3 | HOXD4 | HOXD8 | HOXP | HSAL2 | HSF1 |
| HSFY2 | ID1 | IFNA10 | IFNA13 | IFNA14 | IFNA16 |
| IFNA17 | IFNA2 | IFNA21 | IFNA4 | IFNA5 | IFNA6 |
| IFNA7 | IFNA8 | IFNE | IFNG | IFNW1 | IKZF1 |
| IKZF3 | IL10 | IL11 | IL12B | IL13 | IL15 |
| IL16 | IL17A | IL17C | IL17D | IL17F | IL18 |
| IL19 | IL1A | IL1B | IL1F3 | IL1F5 | IL1F6 |
| IL1F7 | IL1F8 | IL2 | IL20 | IL22 | IL23A |
| IL24 | IL25 | IL26 | IL27 | IL28B | IL31 |
| IL32 | IL33 | IL34 | IL36G | IL4 | IL6 |
| IL7 | IL8 | IL9 | ILF10 | ING2 | ING3 |
| INHA | INHBB | IRF1 | IRF2 | IRF3 | IRF4 |
| IRF5 | IRF6 | IRF8 | IRF9 | IRX2 | IRX3 |
| IRX6 | ISL1 | ISL2 | JDP2 | JUN | JUNB |
| KAT5 | KAT7 | KAY7 | KCNIP2 | KCNMB3 | KDM1 |
| KDM1A | KITLG | KLF1 | KLF10 | KLF11 | KLF12 |
| KLF3 | KLF4 | L3MBTL1 | L3MBTL4 | LAMTOR5 | LASS6 |
| LBX2 | LCOR | LEF1 | LEFTY1 | LEFTY2 | LHX2 |
| LHX4 | LHX6 | LHX9 | LIF | LMO4 | LMX1A |
| LMX1B | LRRC8B | LTA | LTB | MAFB | MAFF |
| MAFG | MARCH5 | MARCKS5 | MAX | MBD1 | MDS1 |
| MECOM | MECP2 | MEF2A | MEF2D | MEIS2 | MEOX1 |
| MICB | MIF | MITA3 | MITF | MLX | MLXIPL |
| MMP14 | MPLKIP | MPO | MSC | MSL3 | MSTN |
| MSX2 | MTA1 | MTA2 | MXD1 | MYB | MYBL2 |
| MYC | MYCL | MYCN | MYF6 | MYOG | MYT1 |
| MZF1 | NAMPT | NANOGP8 | NDOR1 | NDP | NEK6 |
| NEUROD1 | NEUROD2 | NEUROG3 | NFATC1 | NFATC3 | NFE2 |
| NFE2L1 | NFE2L3 | NFIC | NFIL3 | NFKB1 | NFYB |
| NFYC | NKX2-1 | NKX2-3 | NKX2-5 | NKX2-8 | NME2 |
| NOLC1 | NPAS2 | NR0B1 | NR0B2 | NR1D1 | NR1D2 |
| NR1H3 | NR1H3 | NR1I2 | NR1I3 | NR2C2 | NR2E1 |
| NR2E3 | NR2F1 | NR2F2 | NR3C1 | NR4A1 | NR4A2 |
| NR5A1 | NR5A2 | NR6A1 | NRDB2 | NRG1 | NXPE4 |
| OLIG2 | OSM | OTP | OTX1 | OTX2 | PA2G4 |
| PARS2 | PAX4 | PAX5 | PAX6 | PAX8 | PBX2 |
| PCGF6 | PF4 | PFDN1 | PGBD1 | PGRMC2 | PHC2 |
| PHF5A | PHF6 | PHF7 | PHOX2A | PHTF1 | PITX1 |
| PITX2 | PKNOX1 | PKNOX2 | PLAGL2 | POLD4 | POU1F1 |
| POU2F1 | POU2F2 | POU3F2 | POU4F3 | POU6F1 | PPARA |
| PPARG | PPBP | PRDM1 | PRMT5 | PRMT6 | PROP1 |
| PRRX1 | PRRX2 | PTGER3 | PTH | PTTG1 | RARB |
| RARG | RAX2 | RBCK | RBPJ | RBPJL | RCAN1 |
| RELA | RELB | RENIN | RFX3 | RFX5 | RFXANK |
| RFXAP | RHOXF1 | RHOXF2 | RNF4 | RORB | RTP3 |
| RUNX1T1 | RUNX3 | RXRA | RXRB | RXRG | SALL2 |
| SATB2 | SCG2 | SCGB3A1 | SCMH1 | SECTM1 | SETD3 |
| SETD4 | SETD8 | SETDB1 | SHOX | SHOX2 | SIX1 |
| SIX2 | SIX5 | SLC30A9 | SLURP1 | SMAD1 | SMAD2 |
| SMAD3 | SMAD4 | SMAD6 | SNAI2 | SNAPC2 | SNAPC5 |
| SOX10 | SOX13 | SOX2 | SOX5 | SOX6 | SOX7 |
| SOX8 | SOX9 | SP140 | SPDEF | SPI1 | SPIB |
| SPINK13 | SPP1 | SREBF1 | SRF | SRY | STAT1 |
| STAT3 | STAT5 | STAT5A | STAT5B | STAT6 | SUPT4H1 |
| SUV420H1 | TADA2A | TADA3 | TADA3L | TAF10 | TAF12 |
| TAF5L | TAF6 | TAF7 | TARDBP | TBX2 | TBX20 |
| TBX21 | TBX22 | TBX3 | TBX5 | TBX6 | TCEAL1 |
| TCF12 | TCF19 | TCF25 | TCF7 | TCF7L2 | TEAD2 |
| TEAD3 | TEAD4 | TEF | TFAP2C | TFAP2E | TFCP2 |
| TFCP2L1 | TFDP1 | TFDP2 | TFE3 | TFEB | TFEC |
| TGFB2 | TGIF1 | TGIF2 | TGIF2LX | THAPB | THRA |
| THRB | TLE4 | TLX2 | TLX3 | TMEM8C | TMOD1 |
| TNF | TNFRSF11B | TNFSF13 | TNFSF13B | TNFSF15 | TNFSF18 |
| TNFSF4 | TNFSF8 | TNLG8A | TP53 | TP63 | TRAF3IP1 |
| TRIM22 | TRIM25 | TRIM27 | TRIM28 | TRIM29 | TRIM58 |
| TSC22D2 | TSC22D3 | TSC22D3 | TSC22D4 | TSLP | TULP4 |
| TWIST2 | UBP1 | UNC50 | USF1 | USF2 | USP16 |
| USP21 | VAX1 | VAX2 | VDR | VEGFA | VENTX |
| VSX2 | WNT1 | WNT2 | WNT5A | WT1 | XBP1 |
| XCL1 | XCL2 | YBX1 | YEATS4 | YY1 | ZBTB18 |
| ZBTB25 | ZBTB48 | ZEB2 | ZFP29 | ZFP36L1 | ZFP36L2 |
| ZFP42 | ZFP69B | ZHX2 | ZHX3 | ZIC3 | ZKSCAN3 |
| ZKSCAN4 | ZKSCAN5 | ZKSCAN7 | ZKSCAN8 | ZNF131 | ZNF133 |
| ZNF134 | ZNF140 | ZNF165 | ZNF169 | ZNF174 | ZNF175 |
| ZNF18 | ZNF189 | ZNF19 | ZNF192 | ZNF207 | ZNF207 |
| ZNF213 | ZNF215 | ZNF232 | ZNF24 | ZNF256 | ZNF263 |
| ZNF268 | ZNF274 | ZNF277 | ZNF281 | ZNF287 | ZNF34 |
| ZNF354A | ZNF37A | ZNF394 | ZNF396 | ZNF397 | ZNF41 |
| ZNF444 | ZNF446 | ZNF449 | ZNF483 | ZNF496 | ZNF639 |
| ZNF69 | ZNF71 | ZNF75D | ZNF83 | ZNF85 | ZRANB2 |
| ZSCAN1 | ZSCAN12 | ZSCAN16 | ZSCAN18 | ZSCAN20 | ZSCAN21 |
| ZSCAN25 | ZSCAN26 | ZSCAN31 | ZSCAN4 | ZSCAN5A | ZSCAN9 |

TABLE S2
Activators and inhibitors identified by screen from the
human retroviral ORF cDNA library.
Activators

| Gene Symbol | cTnT Z-score | αMHC Z-score |
|---|---|---|
| PHF7 | 9.7 | 7.4 |
| ZNF281 | 7.0 | 2.9 |
| LTA | 6.3 | 0.0 |
| RNF4 | 4.6 | 3.1 |
| SMAD2 | 4.5 | 1.8 |
| TP53 | 4.4 | 1.1 |
| ASCL1 | 3.9 | 0.0 |
| MSX2 | 3.8 | 2.9 |
| MTA1 | 3.8 | 1.7 |
| SHOX2 | 3.6 | 1.6 |
| Nxpe4 | 3.6 | 0.3 |
| TAF6 | 3.5 | 4.0 |
| HHEX | 3.4 | 1.1 |
| TFDP1 | 3.3 | 3.4 |
| SIX2 | 3.3 | 0.0 |
| MYBL2 | 3.3 | 2.9 |
| NR6A1 | 3.2 | 1.7 |
| TCF19 | 3.2 | 1.7 |
| PAX4 | 3.2 | 0.7 |
| NANOGP8 | 3.1 | 1.4 |
| PLAGL2 | 3.0 | 1.5 |
| CREM | 2.9 | 0.3 |
| ELF1 | 2.8 | 1.8 |
| BATF | 2.7 | 0.6 |
| GTF2IRD1 | 2.5 | 7.2 |
| ATF4 | 2.4 | 0.3 |
| CD200R1 | 2.3 | 2.4 |
| HMGB2 | 2.3 | 1.7 |
| CXCL10 | 2.3 | 0.2 |
| AFF4 | 2.2 | 2.0 |
| ATF2 | 2.2 | 1.2 |
| PPARA | 2.2 | 2.2 |
| KITLG | 2.1 | 1.6 |
| NFYC | 2.0 | 0.0 |
| SPP1 | 2.0 | 1.4 |
| CMTM3 | 1.9 | 2.6 |
| TRIM28 | 1.9 | 2.2 |
| ZSCAN26 | 1.6 | 2.0 |
| VENTX | 1.5 | 2.8 |
| AHR | 1.3 | 2.0 |
| IFNA16 | 1.3 | 2.1 |
| IL18 | 1.2 | 2.2 |
| IFNA2 | 1.1 | 2.8 |
| HLF | 0.6 | 2.9 |
| SOX6 | 0.5 | 3.3 |
| MAX | 0.5 | 2.2 |
| IL10 | 0.3 | 2.3 |
| MEF2D | 0.2 | 2.0 |
| LCOR | 0.1 | 2.4 |

Inhibitors

| Gene Symbol | cTnT Z-score | aMHC Z-score |
|---|---|---|
| HOXC4 | −4.2 | −5.6 |
| OLIG2 | −4.1 | −5.4 |
| JUN | −4.0 | −4.1 |
| LHX4 | −3.8 | −3.3 |
| HOXD4 | −3.6 | −4.0 |
| TLE4 | −3.5 | −4.3 |
| POU2F1 | −3.4 | −4.9 |
| HOXB9 | −3.3 | −3.7 |
| DLX3 | −3.2 | −4.0 |
| FOXA3 | −3.1 | −4.3 |
| FOSL1 | −3.0 | 0.4 |
| SRF | −3.0 | −1.5 |
| PHOX2A | −3.0 | −2.6 |
| ZFP36L1 | −3.0 | −1.2 |
| SOX10 | −2.9 | −2.8 |
| HOXD10 | −2.9 | −4.2 |
| LHX9 | −2.8 | −3.4 |
| DBT | −2.8 | −5.0 |
| NFE2 | −2.8 | −2.1 |
| ZNF397 | −2.7 | −3.9 |
| USF2 | −2.7 | −4.8 |
| HNF4A | −2.7 | −4.5 |
| NR5A1 | −2.6 | −4.7 |
| IL2 | −2.6 | −3.7 |
| FUBP1 | −2.5 | −2.9 |
| IRF4 | −2.5 | −3.1 |
| CEBPE | −2.3 | −3.1 |
| NR4A1 | −2.3 | −3.8 |
| AIMP1 | −2.2 | −2.7 |
| PAX6 | −2.2 | −3.2 |
| TLX3 | −2.2 | −2.4 |
| SPDEF | −2.1 | −2.6 |
| FOS | −2.1 | 0.3 |
| SOX9 | −2.1 | −2.0 |
| TGIF2 | −2.1 | −3.6 |
| ZHX2 | −2.1 | −1.7 |
| NR3C1 | −2.0 | −0.6 |
| CTF1 | −2.0 | −1.8 |
| TFEB | −2.0 | −1.0 |
| PITX2 | −2.0 | −2.7 |
| SOX7 | −2.0 | −2.4 |
| HNF1B | −1.9 | −2.2 |
| SNAI2 | −1.9 | −3.7 |
| FASLG | −1.9 | −4.6 |
| HOXA6 | −1.9 | −2.2 |
| OTX1 | −1.8 | −4.3 |
| FAM3B | −1.8 | −2.2 |
| NKX2-5 | −1.8 | −2.8 |
| ESR1 | −1.8 | −5.0 |
| HOXB6 | −1.7 | −2.3 |
| DLX4 | −1.7 | −3.9 |
| TBX2 | −1.7 | −2.8 |
| HOXC6 | −1.6 | −2.9 |
| NR4A2 | −1.6 | −5.3 |
| RHOXF1 | −1.6 | −3.9 |
| TFCP2L1 | −1.6 | −3.3 |
| HOXC5 | −1.6 | −2.7 |
| DLX6 | −1.5 | −2.5 |
| MEOX1 | −1.5 | −3.6 |
| CX3CL1 | −1.5 | −2.5 |
| IL11 | −1.5 | −2.2 |
| HOXA11 | −1.5 | −2.3 |
| HOXD8 | −1.5 | −2.5 |
| SPIB | −1.5 | −2.5 |
| ETS1 | −1.4 | −4.2 |
| TEAD3 | −1.4 | −2.6 |
| HOXB5 | −1.4 | −2.0 |
| DMRT1 | −1.4 | −2.4 |
| HNF1A | −1.4 | −2.3 |
| TNFSF4 | −1.4 | −2.2 |
| RORB | −1.4 | −2.2 |
| ZIC3 | −1.4 | −2.6 |
| IL26 | −1.4 | −3.1 |
| CSRNP3 | −1.4 | −2.9 |
| TARDBP | −1.4 | −2.5 |
| ELF4 | −1.3 | −2.1 |
| HOXB13 | −1.3 | −2.3 |
| CEBPB | −1.3 | −2.1 |
| NR2E1 | −1.3 | −4.1 |
| SMAD3 | −1.3 | −2.1 |
| ALX1 | −1.3 | −3.2 |
| NFIC | −1.3 | −2.0 |
| FOXA1 | −1.3 | −2.1 |
| IKZF3 | −1.3 | −2.1 |
| E4F1 | −1.2 | −2.3 |
| FL1 | −1.2 | −2.1 |
| ERG | −1.2 | −2.1 |
| HOXD3 | −1.2 | −2.2 |
| IL1A | −1.2 | −2.2 |
| PITX1 | −1.2 | −2.7 |
| NEUROD1 | −1.1 | −2.2 |
| ZBTB48 | −1.1 | −2.5 |
| TFAP2E | −1.1 | −2.2 |
| OTP | −1.1 | −3.8 |
| SOX8 | −1.1 | −2.8 |
| MECP2 | −1.1 | −3.9 |
| CREB5 | −1.1 | −2.0 |
| FOXA2 | −1.0 | −3.8 |
| PBX2 | −1.0 | −3.4 |
| DLX1 | −1.0 | −3.8 |
| NKX2-1 | −1.0 | −3.9 |
| PAX5 | −1.0 | −2.1 |
| FOXL2 | −0.9 | −2.3 |
| POU3F2 | −0.9 | −3.1 |
| TBX3 | −0.9 | −3.6 |
| FEV | −0.9 | −2.9 |
| MBD1 | −0.9 | −3.3 |
| TGIF1 | −0.8 | −2.6 |
| ZNF639 | −0.8 | −2.2 |
| PRDM1 | −0.8 | −3.8 |
| POU4F3 | −0.8 | −2.0 |
| EGR2 | −0.7 | −5.3 |
| KLF3 | −0.7 | −2.6 |
| FAM3D | −0.6 | −2.7 |
| MIF | −0.6 | −2.6 |
| ARGFX | −0.4 | −3.5 |
| CMTM2 | −0.4 | −2.4 |
| PRRX2 | 0.0 | −2.1 |
| BMP2 | 0.5 | −3.2 |
| CXCL13 | 0.6 | −2.0 |
| ATOH1 | 1.2 | −2.2 |
| PTGER3 | 1.2 | −2.3 |
| GDF2 | 1.3 | −3.0 |
| PPARG | 1.6 | −4.4 |
| CXCL5 | 2.5 | −3.8 |
| NR0B2 | 2.8 | −2.2 |
| CRX | 7.5 | −3.6 |
| NR2F1 | 9.4 | −2.8 |
| OTX2 | 9.5 | −2.3 |

Genes with Z-scores of αMHC-GFP or cTnT expression ≥ 2 were defined as activators. Genes with Z-scores of αMHC or cTnT expression ≤ −2 were defined as inhibitors.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
“Antibodies: A Laboratory Manual,” Cold Spring Harbor Press, Cold Spring Harbor, NY, 1988.
Abad et al., Stem Cell Reports 8:548-560, 2017.
Ang et al., Cell 167, 1734-1749 e1722, 2016.
Angel et al., Cell, 49:729, 1987a.
Angel et al., Cell, 49:729, 1987b.
Aurora and Olson, Cell Stem Cell 15, 14-25, 2014a.
Aurora and Olson, J Clin Invest 124, 1382-1392, 2014b.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Basson et al., Nat. Genet., 15(1):30-35, 1997.
Benvenisty and Neshif, Proc. Natl. Acad. Sci. USA, 83:9551-9555, 1986.
Berkhout et al., Cell, 59:273-282, 1989.
Bhaysar et al., Genomics, 35(1):11-23, 1996.
Blanar et al., EMBO J., 8:1139, 1989.
Blomer et al., J. Virol., 71(9):6641-6649, 1997.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J Virol., 62:1437, 1986.
Campbell and Villarreal, Mol. Cell. Biol., 8:1993, 1988.
Campbell et al., J. Mol. Biol., 180:1-19, 1984.
Campere and Tilghman, Genes and Dev., 3:537, 1989.
Campo et al., Nature, 303:77, 1983.
Capaldi et al., Biochem. Biophys. Res. Comm., 74(2):425-433, 1977.
Celander and Haseltine, J. Virology, 61:269, 1987.
Celander et al., J Virology, 62:1314, 1988.
Chandler et al., Cell, 33:489, 1983.
Chatterjee et al., Proc. Natl. Acad. Sci. USA, 86:9114, 1989.
Chen and Okayama, Mol. Cell Biol., 7(8):2745-2752, 1987.
Choi et al., Cell, 53:519, 1988.
Cirillo et al., Mol Cell 9, 279-289, 2002.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81-90, 1988.
Coupar et al., Gene, 68:1-10, 1988.
Cripe et al., EMBO J., 6:3745, 1987.
Culotta and Hamer, Mol. Cell. Biol., 9:1376-1380, 1989.
Dandolo et al., J. Virology, 47:55-64, 1983.
De Villiers et al., Nature, 312(5991):242-246, 1984.
Deschamps et al., Science, 230:1174-1177, 1985.
Dubensky et al., Proc. Natl. Acad. Sci. USA, 81:7529-7533, 1984.
Edbrooke et al., Mol. Cell. Biol., 9:1908-1916, 1989.
Edlund et al., Science, 230:912-916, 1985.
Engel and Kohn, Front Biosci, 4:e26-33, 1999.
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Fidalgo et al., Proceedings of the National Academy of Sciences of the United States of America 109, 16202-16207, 2012.
Fidalgo et al., Cell stem cell 19, 355-369, 2016.
Fidalgo et al., Stem Cells 29, 1705-1716, 2011.
Fischer, Med. Res. Rev., 27(6):755-796, 2007.
Foecking and Hofstetter, Gene, 45(1):101-105, 1986.
Fraley et al., Proc. Natl. Acad. Sci. USA, 76:3348-3352, 1979.
Franz et al., Cardoscience, 5(4):235-43, 1994.
Friedmann, Science, 244:1275-1281, 1989.
Fu et al., Stem Cell Reports 1, 235-247, 2013.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Gomez-Del Arco et al., Cell Metab 23, 881-892, 2016.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Graham and van der Eb, Virology, 52:456-467, 1973.
Greene et al., Immunology Today, 10:272, 1989
Grosschedl and Baltimore, Cell, 41:885, 1985.
Grunhaus and Horwitz, Seminar in Virology, 3:237-252, 1992.
Hahn et al., J Mol Med (Berl) 92, 571-581, 2014.
Hanada et al., Biochim. Biophys. Acta, 1697:3-16, 2004.
Harland and Weintraub, J. Cell Biol., 101(3):1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hersdorffer et al., DNA Cell Biol., 9:713-723, 1990.
Hirochika et al., J. Virol., 61:2599, 1987.
Hitraya et al., Arthritis Rheum. 41(11):2048-58 1998.
Holbrook et al., Virology, 157:211, 1987.
Horlick and Benfield, Mol. Cell. Biol., 9:2396, 1989.
Horwich et al. J. Virol., 64:642-650, 1990.
Hsieh et al., Nat. Med., 13:970-974, 2007.
Huang et al., Cell, 27:245, 1981.
Huang et al., Science 338, 1599-1603, 2012.
Hug et al., Mol. Cell. Biol., 8:3065-3079, 1988.
Hwang et al., Mol. Cell. Biol., 10:585, 1990.
Ieda et al., Cell 142, 375-386, 2010.
Ifkovits, PLoS One 9, e89678, 2014.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jessup et al., N Engl J Med 348, 2007-2018 2003.
Kang et al., Science 349, aaa5612, 2015.
Joseph et al., Cancer Cell 13(2):129-40, 2008.
Johnson et al., Mol. Cell. Biol., 9(8):3393-3399, 1989.
Kadesch and Berg, Mol. Cell. Biol., 6:2593, 1986.
Kaneda et al., Science, 243:375-378, 1989.
Kao et al., Genes Dev., 14:55-66, 2000.
Karin et al., Mol. Cell. Biol., 7:606, 1987.
Katinka et al., Cell, 20:393, 1980.
Kato et al., J. Biol Chem., 266(6):3361-3364, 1991.
Kay, Haemophilia, 4(4):389-392, 1998.
Kelly et al., J. Cell Biol., 129(2):383-96, 1995.
Kiledjian et al., Mol. Cell. Biol., 8:145, 1988.
Kimura et al., Dev. Growth Differ., 39(3):257-65, 1997.
King et al., J. Biol. Chem., 269, 10210-10218, 1989.
Klamut et al., Mol. Cell. Biol., 10:193, 1990.
Klein et al., Nature, 327:70-73, 1987.
Koch et al., Mol. Cell. Biol., 9:303, 1989.
Kohut et al., Am. J. Physiol., 275(6Pt1):L1089-1094, 1998.
Kojima et al., Cell Mol Life Sci 74, 2203-2215, 2017.
Kooby et al., FASEB J, 13(11):1325-34, 1999.
Kraus et al. FEBS Lett., 428(3):165-170, 1998.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kuhl et al., Cell, 50:1057, 1987.
Kunz et al., Nucl. Acids Res., 17:1121, 1989.
Lai et al., Elife 6, 2017.
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Laughlin et al., J Virol., 60(2):515-524, 1986.
Lebkowski et al., Mol. Cell. Biol., 8(10):3988-3996, 1988.
Lee et al., Nature, 294:228, 1981.
Lee et al., Cell 151, 547-558, 2012.
Leibowitz et al., Diabetes, 48(4):745-753, 1999.
Leiper et al., Baillieres Clin. Gastroenterol., 12(1):179-199, 1998.
Levinson et al., Nature, 295:79, 1982.
Lin et al., Mol. Cell. Biol., 10:850, 1990.
Luna-Zurita et al., Cell 164, 999-1014, 2016.
Lundstrom, J. Recept Signal Transduct. Res., 19(1-4):673-686, 1999.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
Markowitz et al., J Virol., 62:1120-1124, 1988.
McDermott et al., Mol. Cell. Biol., 13(4):2564-2577, 1993.
McLaughlin et al., J Virol., 62(6):1963-1973, 1988.
McLean et al., Nat Biotechnol 28, 495-501, 2010.
McNeall et al., Gene, 76:81, 1989.
Miksicek et al., Cell, 46:203, 1986.
Mohamed et al., Circulation 135, 978-995, 2017.
Molkentin et al., Mol. Cell. Biol., 16(6):2627-2636, 1996.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Mouse et al., Genome Biol 13, 418, 2012.
Muesing et al., Cell, 48:691, 1987.
Muraoka et al., EMBO J 33, 1565-1581, 2014.
Muzyczka, Curr. Topics Microbiol. Immunol., 158:97-129, 1992.
Naldini et al., Science, 272(5259):263-267, 1996.
Nam et al., Proc Natl Acad Sci USA 110, 5588-5593, 2013.
Ng et al., Nuc. Acids Res., 17:601, 1989.
Nicolas and Rubinstein, Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Okada et al., American Journal of Physiology: Renal Physiology, 275 (2): F306-F314, 1998.
Ondek et al., EMBO J, 6:1017, 1987.
Ornitz et al., Mol. Cell. Biol., 7:3466, 1987.
Palmiter et al., Cell, 29:701, 1982.
Pech et al., Mol. Cell. Biol., 9:396, 1989.
Pelletier and Sonenberg, Nature, 334:320-325, 1988.
Perales et al., Proc. Natl. Acad. Sci. USA, 91(9):4086-4090, 1994.
Perez-Stable and Constantin, Mol. Cell. Biol., 10:1116, 1990. Physicians Desk Reference.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Qian et al., Nature 485, 593-598, 2012.
Queen and Baltimore, Cell, 35:741, 1983.
Quinn et al., Mol. Cell. Biol., 9:4713, 1989.
Ramirez et al., Nucleic Acids Res 44, W160-165, 2016.
Ramirez-Carrozzi et al., Genes Dev 20, 282-296, 2006.
Reisman and Rotter, Mol. Cell. Biol., 9:3571, 1989.
Remington's Pharmaceutical Sciences, 15^{th} ed., 1035-1038 and 1570-1580, Mack Publishing Company, P A, 1980.
Resendez Jr. et al., Mol. Cell. Biol., 8:4579, 1988.
Ridgeway, In: Vectors: A Survey of Molecular Cloning Vectors and Their Uses, Rodriguez et al. (Eds.), Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Russell et al., Biochim. Biophys. Acta, 1443(3):393-399, 1999.
Sakai et al., Genes and Dev., 2:1144, 1988.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Smith et al., Neuron., 20:1093-1102, 1998.
Song et al., Nature 485, 599-604, 2012.
Soufi et al., Cell 161, 555-568, 2015.
Spalholz et al., Cell, 42:183, 1985.
Spandau and Lee, J. Virology, 62:427, 1988.
Spandidos and Wilkie, EMBO J., 2:1193, 1983.
Srivastava et al., Cell 166, 1386-1396, 2016.
Stephens and Hentschel, Biochem. 1, 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takahashi et al., Antibiotics, 49:453, 1996.
Takebe et al., Mol. Cell. Biol., 8:466, 1988.
Tallquist and Molkentin, Nat Rev Cardiol. 14(8):484-491, 2017.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al. J. Biol. Chem., 264:15160, 1989.
Temin, In: Gene Transfer, Kucherlapati (Ed.), NY, Plenum Press, 149-188, 1986.
Terrett et al., Nat. Genet., 6(4):401-404, 1994.
The Merck Index, 11th Edition.
Thiesen et al., J. Virology, 62:614, 1988.
Tratschin et al., Mol. Cell. Biol., 4:2072-2081, 1984.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tur-Kaspa et al., Mol. Cell Biol., 6:716-718, 1986.
U.S. Pat. No. 5,139,941
U.S. Pat. No. 5,580,859
U.S. Pat. No. 5,670,488
U.S. Pat. No. 5,672,681
U.S. Pat. No. 5,994,136
U.S. Pat. No. 6,013,516
Vannice and Levinson, J. Virology, 62:1305, 1988.
Varmus et al., Cell, 25:23-36, 1981.
Vaseghi et al., Protein Cell, 2017.
Vasseur et al., Proc Natl. Acad. Sci. USA., 77:1068, 1980.
Wagner et al., Proc. Natl. Acad. Sci. USA 87(9):3410-3414, 1990.
Wang and Calame, Cell, 47:241, 1986.
Wang et al., Infect. Immun., 66:4193-202, 1998.
Wang et al., Circ Res 116, 237-244, 2015.
Weber et al., Cell, 36:983, 1984.
Weinberger et al. Mol. Cell. Biol., 8:988, 1984.
White et al., Genomics, 27(1):20-26, 1995.
Wong et al., Gene, 10:87-94, 1980.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Xin et al., Nat Rev Mol Cell Biol 14, 529-541, 2013.
Yamakawa et al., Stem Cell Reports 5, 1128-1142, 2015.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yang et al., Dev Cell 22, 1041-1051, 2012.
Young et al., In: Handbook of Applied Therapeutics, 7.1-7.12 and 9.1-9.10, 1989.
Yutzey et al. Mol. Cell. Biol., 9:1397, 1989.
Zelenin et al., FEBS Lett., 280:94-96, 1991.
Zhang et al., Cell, 110:479-488, 2002.
Zhao et al., Nat Commun 6, 8243, 2015.
Zhao-Emonet et al., Biochim. Biophys. Acta, 1442(2-3):109-119, 1998.
Zhou et al., Nature, 361(6412):543-547, 1993.
Zhou et al., Proc. Natl. Acad. Sci. USA, 98:10572-10577, 2001.
Zhou et al., Proc Natl Acad Sci USA 112, 11864-11869, 2015.
Zhou et al., Cell Stem Cell 18, 382-395, 2016.
Ziober and Kramer, J. Bio. Chem., 271(37):22915-22922, 1996.
Zufferey et al., Nat. Biotechnol., 15(9):871-875, 1997.

1. A method of reprogramming a cardiac fibroblast comprising contacting said cardiac fibroblast with AKT1, GATA4, TBX5, MEF2C, HAND2 and ZNF281.

2. The method of claim 1, wherein contacting comprises delivering AKT1, GATA4, TBX5, MEF2C, HAND2 and ZNF281 proteins to said cardiac fibroblast.

3. The method of claim 2, wherein one or more of AKT1, GATA4, TBX5, MEF2C, HAND2 and ZNF281 comprise a heterologous cell permeability peptide (CPP).

4. The method of claim 2, further comprising contacting said cardiac fibroblast with an anti-inflammatory agent and/or with myocardin.

5-6. (canceled)

7. The method of claim 1, contacting comprises delivering AKT1, GATA4, TBX5, MEF2C, HAND2 and ZNF281 expression cassettes to said cardiac fibroblasts.

8. The method of claim 7, wherein said expression cassettes are comprised in one or more replicable vectors.

9. The method of claim 8, wherein said one or more replicable vectors are one or more viral vectors.

10. The method of claim 9, wherein said one or more viral vectors are one or more adeno-associated virus (AAV), non-integrated lentivirus, adenoviral vectors or retroviral vectors.

11. The method of claim 8, wherein said one or more replicable vectors are non-viral vectors.

12. The method of claim 11, wherein said one or more non-viral vectors are disposed in a lipid delivery vehicle.

13. The method of claim 7, further comprising contacting said cardiac fibroblast with an anti-inflammatory agent.

14. The method of claim 7, further comprising contacting said cardiac fibroblast with a myocardin expression cassette.

15. The method of claim 13, further comprising contacting said cardiac fibroblast with a myocardin expression cassette.

16. A method of treating a subject having suffered a myocardial infarct (MI) comprising delivering to said subject AKT1, GATA4, TBX5, MEF2C, HAND2 and ZNF281.

17. The method of claim 16, wherein delivering comprises administration of AKT1, GATA4, TBX5, MEF2C, HAND2 and ZNF281 proteins to said subject.

18. The method of claim 17, wherein one or more of AKT1, GATA4, TBX5, MEF2C, HAND2 and ZNF281 comprise a heterologous cell permeability peptide (CPP).

19. The method of claim 17, further comprising delivering to said subject an anti-inflammatory agent.

20. The method of claim 16, delivering comprises administration of AKT1, GATA4, TBX5, MEF2C, HAND2 and ZNF281 expression cassettes to said subject.

21. The method of claim 20, further comprising delivering to said subject an anti-inflammatory agent.

22. The method of claim 20, wherein said expression cassettes are comprised in replicable vectors.

23. The method of claim 22, wherein said replicable vectors are viral vectors.

24. The method of claim 23, wherein said viral vectors are adeno-associated virus (AAV), non-integrated lentivirus, adenoviral vectors or retroviral vectors.

25. The method of claim 22, wherein said replicable vectors are non-viral vectors.

26. The method of claim 25, wherein said non-viral vectors are disposed in a lipid delivery vehicle.

27. The method of claim 16, wherein AKT1, GATA4, TBX5, MEF2C, HAND2 and ZNF281 proteins or expression cassettes are delivered 24 hours to one month following said MI.

28. The method of claim 16, further comprising delivering myocardin or an expression cassette coding therefore to said subject.

29. The method of claim 17, wherein at least one of said AKT1, GATA4, TBX5, MEF2C, HAND2 and ZNF281 proteins are delivered multiple times.

30. The method of claim 20, wherein at least one of said AKT1, GATA4, TBX5, MEF2C, HAND2 and ZNF281 expression cassettes are delivered multiple times.

31-32. (canceled)

33. The method of claim 16, wherein AKT1, GATA4, TBX5, MEF2C, HAND2 and ZNF281 are delivered daily.

34. The method of claim 33, further comprising delivering myocardin or myocardin expression cassette daily.

35. The method of claim 16, wherein AKT1, GATA4, TBX5, MEF2C, HAND2 and ZNF281 proteins or expression cassettes are delivered via intracardiac injection.

36. The method of claim 16, wherein said subject is further administered oxygen, aspirin, and/or nitroglycerin.

37. The method of claim 16, wherein said subject is further administered percutaneous coronary intervention.

38. The method of claim 16, wherein said subject is further administered a fibrinolytic.

39. The method of claim 16, wherein said MI is non-ST-elevated MI.

40. The method of claim 16, wherein said MI is ST-elevated MI.

41. A method preventing or delaying development of cardiac hypertrophy or heart failure in a subject having suffered a myocardial infarct (MI) comprising providing to said subject AKT1, GATA4, TBX5, MEF2C, HAND2 and ZNF281 proteins or expression cassettes coding therefor.

42. The method of claim 41, further comprising administering to said subject a secondary anti-hypertrophic or heart failure therapy.

43. The method of claim 41, wherein the secondary therapy is a PKD inhibitor, a beta blocker, an ionotrope, a diuretic, ACE-I, All antagonist, BNP, a Ca⁺⁺-blocker, or an HDAC inhibitor.

44. The method of claim 41, wherein preventing or delaying comprises preventing or delaying cardiac hypertrophy.

45. The method of claim 41, wherein preventing or delaying comprises preventing or delaying one or more of decreased exercise capacity, decreased cardiac ejection volume, increased left ventricular end diastolic pressure, increased pulmonary capillary wedge pressure, decreased cardiac output or cardiac index, increased pulmonary artery pressures, increased left ventricular end systolic and diastolic dimensions, increased left and right ventricular wall stress, increased wall tension, decreased quality of life, and/or increased disease related morbidity or mortality.

46. The method of claim 41, wherein AKT1, GATA4, TBX5, MEF2C, HAND2 and ZNF281 proteins are administered to said subject.

47. The method of claim 41, wherein AKT1, GATA4, TBX5, MEF2C, HAND2 and ZNF281 expression cassettes are administered to said subject.

48. The method of claim 46, further comprising administering myocardin protein or expression cassette coding therefore and/or an anti-inflammatory agent to said subject.

49. (canceled)

50. A method of reducing a decrease in exercise tolerance of a subject having suffered a myocardial infarction comprising administering to said subject AKT1, GATA4, TBX5, MEF2C, HAND2 and ZNF281 proteins or expression cassettes coding therefor.

51. The method of claim 50, further comprising administering myocardin or an expression cassette coding therefor to said subject.

52. A method of reducing hospitalization of a subject having suffered a myocardial infarction comprising administering to said subject AKT1, GATA4, TBX5, MEF2C, HAND2 and ZNF281 proteins or expression cassettes coding therefor.

53. (canceled)

54. A method of improving quality of life of a subject having suffered a myocardial infarction comprising administering to said subject AKT1, GATA4, TBX5, MEF2C, HAND2 and ZNF281 proteins or expression cassettes coding therefor.

55. (canceled)

56. A method of decreasing morbidity of a subject having suffered a myocardial infarction comprising administering to said subject AKT1, GATA4, TBX5, MEF2C, HAND2 and ZNF281 proteins or expression cassettes coding therefor.

57. (canceled)

58. A method of decreasing mortality of a subject having suffered a myocardial infarction comprising administering to said subject AKT1, GATA4, TBX5, MEF2C, HAND2 and ZNF281 proteins or expression cassettes coding therefor.

59-60. (canceled)

61. A method of reprogramming a cardiac fibroblast comprising contacting said cardiac fibroblast with AKT1, GATA4, TBX5, MEF2C, HAND2 and ASCL1.

62. The method of claim 61, wherein contacting comprises delivering AKT1, GATA4, TBX5, MEF2C, HAND2 and ASCL1 proteins to said cardiac fibroblast.

63. The method of claim 62, wherein one or more of AKT1, GATA4, TBX5, MEF2C, HAND2 and ASCL1 comprise a heterologous cell permeability peptide (CPP).

64. The method of claim 62, further comprising contacting said cardiac fibroblast with an anti-inflammatory agent and/or mocardin.

65-66. (canceled)

67. The method of claim 61, contacting comprises delivering AKT1, GATA4, TBX5, MEF2C, HAND2 and ASCL1 expression cassettes to said cardiac fibroblasts.

68. The method of claim 67, wherein said expression cassettes are comprised in one or more replicable vectors.

69. The method of claim 68, wherein said one or more replicable vectors are one or more viral vectors.

70. The method of claim 69, wherein said one or more viral vectors are one or more adeno-associated virus (AAV), non-integrated lentivirus, adenoviral vectors or retroviral vectors.

71. The method of claim 68, wherein said one or more replicable vectors are non-viral vectors.

72. The method of claim 71, wherein said one or more non-viral vectors are disposed in a lipid delivery vehicle.

73. The method of claim 67, further comprising contacting said cardiac fibroblast with an anti-inflammatory agent.

74. The method of claim 67, further comprising contacting said cardiac fibroblast with a myocardin expression cassette.

75. The method of claim 73, further comprising contacting said cardiac fibroblast with a myocardin expression cassette.

76. A method of treating a subject having suffered a myocardial infarct (MI) comprising delivering to said subject AKT1, GATA4, TBX5, MEF2C, HAND2 and ASCL1.

77. The method of claim 76, wherein delivering comprises administration of AKT1, GATA4, TBX5, MEF2C, HAND2 and ASCL1 proteins to said subject.

78. The method of claim 77, wherein one or more of AKT1, GATA4, TBX5, MEF2C, HAND2 and ASCL1 comprise a heterologous cell permeability peptide (CPP).

79. The method of claim 77, further comprising delivering to said subject an anti-inflammatory agent.

80. The method of claim 76, delivering comprises administration of AKT1, GATA4, TBX5, MEF2C, HAND2 and ASCL1 expression cassettes to said subject.

81. The method of claim 80, further comprising delivering to said subject an anti-inflammatory agent.

82. The method of claim 80, wherein said expression cassettes are comprised in replicable vectors.

83. The method of claim 82, wherein said replicable vectors are viral vectors.

84. The method of claim 83, wherein said viral vectors are adeno-associated virus (AAV), non-integrated lentivirus, adenoviral vectors or retroviral vectors.

85. The method of claim 82, wherein said replicable vectors are non-viral vectors.

86. The method of claim 85, wherein said non-viral vectors are disposed in a lipid delivery vehicle.

87. The method of claim 76, wherein AKT1, GATA4, TBX5, MEF2C, HAND2 and ASCL1 proteins or expression cassettes are delivered 24 hours to one month following said MI.

88. The method of claim 76, further comprising delivering myocardin or an expression cassette coding therefore to said subject.

89. The method of claim 77, wherein at least one of said AKT1, GATA4, TBX5, MEF2C, HAND2 and ASCL1 proteins are delivered multiple times.

90. The method of claim 80, wherein at least one of said AKT1, GATA4, TBX5, MEF2C, HAND2 and ASCL1 expression cassettes are delivered multiple times.

91-92. (canceled)

93. The method of claim 76, wherein AKT1, GATA4, TBX5, MEF2C, HAND2 and ASCL1 are delivered daily.

94. The method of claim 93, further comprising delivering myocardin or myocardin expression cassette daily.

95. The method of claim 76, wherein AKT1, GATA4, TBX5, MEF2C, HAND2 and ASCL1 proteins or expression cassettes are delivered via intracardiac injection.

96. The method of claim 76, wherein said subject is further administered oxygen, aspirin, and/or nitroglycerin.

97. The method of claim 76, wherein said subject is further administered percutaneous coronary intervention.

98. The method of claim 76, wherein said subject is further administered a fibrinolytic.

99. The method of claim 76, wherein said MI is non-ST-elevated MI.

100. The method of claim 76, wherein said MI is ST-elevated MI.

101. A method preventing or delaying development of cardiac hypertrophy or heart failure in a subject having suffered a myocardial infarct (MI) comprising providing to said subject AKT1, GATA4, TBX5, MEF2C, HAND2 and ASCL1 proteins or expression cassettes coding therefor.

102. The method of claim 101, further comprising administering to said subject a secondary anti-hypertrophic or heart failure therapy.

103. The method of claim 101, wherein the secondary therapy is a PKD inhibitor, a beta blocker, an ionotrope, a diuretic, ACE-I, All antagonist, BNP, a Ca⁺⁺-blocker, or an HDAC inhibitor.

104. The method of claim 101, wherein preventing or delaying comprises preventing or delaying cardiac hypertrophy.

105. The method of claim 101, wherein preventing or delaying comprises preventing or delaying one or more of decreased exercise capacity, decreased cardiac ejection volume, increased left ventricular end diastolic pressure, increased pulmonary capillary wedge pressure, decreased cardiac output or cardiac index, increased pulmonary artery pressures, increased left ventricular end systolic and diastolic dimensions, increased left and right ventricular wall stress, increased wall tension, decreased quality of life, and/or increased disease related morbidity or mortality.

106. The method of claim 101, wherein AKT1, GATA4, TBX5, MEF2C, HAND2 and ASCL1 proteins are administered to said subject.

107. The method of claim 101, wherein AKT1, GATA4, TBX5, MEF2C, HAND2 and ASCL1 expression cassettes are administered to said subject.

108. The method of claim 106, further comprising administering myocardin protein or expression cassette coding therefore to said subject.

109. The method of claim 107, further comprising administering an anti-inflammatory agent to said subject.

110. A method of reducing a decrease in exercise tolerance of a subject having suffered a myocardial infarction comprising administering to said subject AKT1, GATA4, TBX5, MEF2C, HAND2 and ASCL1 proteins or expression cassettes coding therefor.

111. (canceled)

112. A method of reducing hospitalization of a subject having suffered a myocardial infarction comprising administering to said subject AKT1, GATA4, TBX5, MEF2C, HAND2 and ASCL1 proteins or expression cassettes coding therefor.

113. (canceled)

114. A method of improving quality of life of a subject having suffered a myocardial infarction comprising administering to said subject AKT1, GATA4, TBX5, MEF2C, HAND2 and ASCL1 proteins or expression cassettes coding therefor.

115. (canceled)

116. A method of decreasing morbidity of a subject having suffered a myocardial infarction comprising administering to said subject AKT1, GATA4, TBX5, MEF2C, HAND2 and ASCL1 proteins or expression cassettes coding therefor.

117. (canceled)

118. A method of decreasing mortality of a subject having suffered a myocardial infarction comprising administering to said subject AKT1, GATA4, TBX5, MEF2C, HAND2 and ASCL1 proteins or expression cassettes coding therefor.

119. (canceled)

120. The method of claim 110, further comprising administering and anti-inflammatory agent to said subject.
